(19) United States
(12) Patent Application Publication
(10) Pub. No.: US 2012/0077990 A1
(43) Pub. Date: Mar. 29, 2012

(54) Alkene Aziridination

(76) Inventor: X. Peter Zhang, Tampa, FL (US)

(21) Appl. No.: 13/202,804

(22) PCT Filed: Feb. 24, 2010
(86) PCT No.: PCT/US10/25240

§ 371 Date: Nov. 8, 2011

Related U.S. Application Data
(60) Provisional application No. 61/155,004, filed on Feb. 24, 2009.

(51) Int. Cl. C07D 203/24; C07D 203/26; C07C 247/10

(52) U.S. Cl. 548/961; 548/965; 552/5

(57) ABSTRACT

A process for the asymmetric aziridination of an alkene comprising treating the alkene with a sulfonyl azide, preferably trichloroethoxysulfonyl azide, in the presence of a cobalt(II) porphyrin.

### CROSS-REFERENCE TO RELATED APPLICATIONS

This application claims priority to U.S. Provisional Application Ser. No. 61/155,004, filed Feb. 24, 2009, which is incorporated herein by reference in its entirety.

### STATEMENT OF GOVERNMENT RIGHTS

This invention was made with Government support under grant number NSF 0711024 awarded by the National Science Foundation. The government has certain rights in the invention.

### FIELD OF THE INVENTION

The present invention generally relates to metal-catalyzed aziridination of alkenes. More particularly, the present invention relates to a process for asymmetric aziridination of alkenes using a metal porphyrin complex catalyst and an azide.

### BACKGROUND OF THE INVENTION

Aziridines, the smallest nitrogen heterocyclic compounds, exhibit numerous important applications, including serving as essential motifs of biologically interesting compounds and as valuable synthons for preparation of various amine derivatives. See, for example, Hu, Tetrahedron 2004, 60, 2701; Sweeney, Chem. Soc. Rev. 2002, 31, 247; Zwanenburg et al., Top. Curr. Chem. 2001, 216, 93; and McCoull et al., Synthesis 2000, 1347. Among several approaches, metal-catalyzed asymmetric aziridination of alkenes with proper nitrene sources represents one of the most general and direct methods for stereoselective construction of the three-membered ring structure. See, for example, Muller et al., Chem. Rev. 2003, 103, 2905; Jacobsen, In Comprehensive Asymmetric Catalysis; Jacobsen et al., Eds.; Springer: Berlin, 1999, 2, 607; and Halfen, Curr. Org. Chem. 2005, 9, 657.

As the common nitrene source, iminoiodane derivatives such as PhI═NTs and its variants have been successfully employed for asymmetric aziridination of certain types of alkenes in catalytic systems that are primarily based on chiral Cu complexes. For select examples of asymmetric aziridination of alkenes, see Li et al., J. Am. Chem. Soc. 1993, 115, 5326; Evans et al., J. Am. Chem. Soc. 1993, 115, 5328; Sanders et al., J. Am. Chem. Soc. 2000, 122, 7132; Wang et al., Chem. Eur. J. 2006, 12, 4568; Nishikori et al., Tetrahedron Lett. 1996, 37, 9245; Muller et al., Tetrahedron 1996, 52, 1543; Liang et al., Chem. Commun. 2002, 124; Anada et al., Org. Lett., 2007, 9, 43559; and Gruit et al., Tetrahedron: Asymmetry, 2004, 15, 1019. While some of the most promising Cu-catalyzed systems can efficiently aziridinate internal alkenes having additional functionalities (such as cinnamate esters) with high enantioselectivity, they are significantly less effective for simple terminal olefins (such as styrenes), suggesting the requirement of secondary binding interactions. Despite the perceived ineffectiveness of azides for metal-catalyzed nitrene transfer reactions, recent results from several groups suggest the potential of azides as a class of general nitrene sources with several attractive features. Fantauzzi et al., Organometallics. 2005, 24, 4710; Piangiolino et al., Eur. J. Org. Chem. 2007, 743; Fantauzzi et al., Eur. J. Org. Chem., 2007, 6053; Caselli et al., J. Org. Chem., 2008, 3009; Omura et al., Chem. Lett. 2003, 354; Omura et al., Chem. Commun. 2004, 2060; Kawabata et al., Tetrahedron Lett. 2006, 47, 1571; Kawabata et al., T. Chem. Asian J. 2007, 2, 248; Stokes et al., J. Am. Chem. Soc. 2007, 129, 7500; Shen et al., Angew. Chem., Int. Ed., 2008, 47, 5056; Badiei et al., Angew. Chem. Int. Ed., 2008, 47, 9961; Han et al., J. Org. Chem., 2008, 73, 2862; Katsuki, Chem. Lett., 2005, 1304; Cenini et al., Coor. Chem. Rev., 2006, 250, 1234. Through the discovery of new chiral catalysts, asymmetric aziridination with azides may be further developed into a broad and useful catalytic process.

We recently disclosed the unique catalytic properties of Co(II) complexes of porphyrins for olefin aziridination and C—H amination with different azides, including diarylphosphoryl and arylsulfonyl azides. Gao et al., J. Org. Chem. 2006, 71, 6655; Ruppel et al., Org. Lett. 2007, 9, 4889; Ruppel et al., Org. Lett. 2008, 10, 1995; and Jones et al., J. Org. Chem., 2008, 73, 7260. To improve catalytic efficacy and to control enantioselectivity, efforts have been made to identify more effective azides and to employ suitable chiral porphyrin ligands for the development of Co-catalyzed asymmetric aziridination with azides.

### SUMMARY OF THE INVENTION

The present invention provides for a general and efficient catalytic system for asymmetric aziridination of alkenes. Among the various aspects of the present invention, therefore, is a process for asymmetric aziridination of an alkene with a metal porphyrin complex, preferably a cobalt(II) complex, with a sulfonyl azide. D₂-symmetric chiral cobalt (II) complexes can catalyze the aziridination of alkenes, forming the corresponding aziridines in high yields and selectivities. Herein, we report a highly asymmetric aziridination process that, in a preferred embodiment, consists of trichloroethoxysulfonyl azide (TcesN₃) as a new nitrene source and Co(II) complexes of D₂-symmetrical chiral porphyrins ([Co(Por*)]) as catalysts.

The [Co(Por*)]/TcesN₃-based catalytic system is operationally simple and capable of aziridinating both aromatic and aliphatic olefins under mild conditions, forming the corresponding N-Tces-aziridines in high yields and excellent enantioselectivities. Advantageously, the catalytic system could be conveniently recycled and reused multiple times through a simple precipitation/filtration protocol without significant loss of reactivity and selectivity. In addition, a noteworthy additive effect of Pd(OAc)₂ on the yield of the Co-catalyzed aziridination is also described.

One aspect of the present invention, therefore, is a process for the asymmetric aziridination of an alkene, the process comprising treating the alkene with a sulfonyl azide in the presence of a cobalt(II) porphyrin complex, the sulfonyl azide having the formula

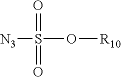

wherein R₁₀ is hydrocarbyl, substituted hydrocarbyl, or heterocyclo.

The present invention is further directed to a process for asymmetric aziridination of an alkene with trichloroethoxysulfonyl azide.

The present invention is further directed to optionally substituted hydrocarbyloxysulfonyl azide. In a preferred embodiment, the optionally substituted hydrocarbyloxysulfonyl azide is trichloroethoxysulfonyl azide.

Other objects and features will be in part apparent and in part pointed out hereinafter.

### ABBREVIATIONS AND DEFINITIONS

The following definitions and methods are provided to better define the present invention and to guide those of ordinary skill in the art in the practice of the present invention. Unless otherwise noted, terms are to be understood according to conventional usage by those of ordinary skill in the relevant art.

The term “acyl,” as used herein alone or as part of another group, denotes the moiety formed by removal of the hydroxyl group from the group —COOH of an organic carboxylic acid, e.g., RC(O)—, wherein R is R¹, R¹O—, R¹R²N— or R¹S—, R¹ is hydrocarbyl, heterosubstituted hydrocarbyl, or heterocyclo and R² is hydrogen, hydrocarbyl or substituted hydrocarbyl.

The term “acyloxy,” as used herein alone or as part of another group, denotes an acyl group as described above bonded through an oxygen linkage (—O—), e.g., RC(O)O— wherein R is as defined in connection with the term “acyl.”

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

The terms “alkoxy” or “alkoxyl” as used herein alone or as part of another group denote any univalent radical, RO— where R is an alkyl group.

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl, and the like. The substituted alkyl groups described herein may have, as substituents, any of the substituents identified as substituted hydrocarbyl substituents.

Unless otherwise indicated, the alkynyl groups described herein are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The terms “aryl” or “ar” as used herein alone or as part of another group denote optionally substituted homocyclic aromatic groups, preferably monocyclic or bicyclic groups containing from 6 to 12 carbons in the ring portion, as phenyl, biphenyl, naphthyl, substituted phenyl, substituted biphenyl or substituted naphthyl. Phenyl and substituted phenyl are the more preferred aryl. The substituted aryl groups described herein may have, as substituents, any of the substituents identified as substituted hydrocarbyl substituents.

The terms “diazo” or “azo” as used herein alone or as part of another group denote an organic compound with two linked nitrogen compounds. These moieties include without limitation diazomethane, ethyl diazoacetate, and t-butyl diazoacetate.

The term “electron acceptor” as used herein denotes a chemical moiety that accepts electrons. Stated differently, an electron acceptor is a chemical moiety that accepts either a fractional electronic charge from an electron donor moiety to form a charge transfer complex, accepts one electron from an electron donor moiety in a reduction-oxidation reaction, or accepts a paired set of electrons from an electron donor moiety to form a covalent bond with the electron donor moiety.

The terms “halogen” or “halo” as used herein alone or as part of another group denote chlorine, bromine, fluorine, and iodine.

The term “heteroaromatic” as used herein alone or as part of another group denotes optionally substituted aromatic groups having at least one heteroatom in at least one ring, and preferably 5 or 6 atoms in each ring. The heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxyl, protected hydroxyl, acyl, acyloxy, alkoxy, alkenoxy, alkynoxy, aryloxy, halogen, amido, amino, nitro, cyano, thiol, ketals, acetals, esters and ethers.

The term “heteroatom” as used herein denotes atoms other than carbon and hydrogen.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainded of the molecule through a carbon or heteroatom. Exemplary heterocyclo include heteroaromatics as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxyl, protected hydroxyl, acyl, acyloxy, alkoxy, alkenoxy, alkynoxy, aryloxy, halogen, amido, amino, nitro, cyano, thiol, ketals, acetals, esters and ethers.

The terms “hydrocarbon” and “hydrocarbyl” as used herein alone or as part of another group denote organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, as alkaryl, alkenaryl, and alkynaryl. Unless otherwise indicated, these moieties preferably comprise 1 to 20 carbon atoms.

The term porphyrin refers to a compound comprising a fundamental skeleton of four pyrrole nuclei united through the α-positions by four methane groups to form the following macrocyclic structure:

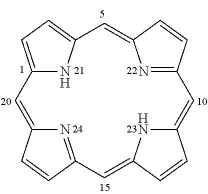

The “substituted hydrocarbyl” moieties described herein, e.g., the substituted alkyl, the substituted alkenyl, the substituted alkynyl, and the substituted aryl moieties, are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substitutents include halogen, heterocyclo, alkoxy, alkenoxy, alkynoxy, aryloxy, hydroxyl, protected hydroxy, keto, acyl, acyloxy, nitro, amino, amido, nitro, cyano, thiol, ketals, acetals, esters and ethers.

### DETAILED DESCRIPTION OF THE PREFERRED EMBODIMENTS

One aspect of the present invention is directed to a process for the preparation of chiral aziridines in diastereo- and enantioenriched form. In general, the process comprises treating an alkene with an optionally substituted hydrocarbyloxysulfonyl azide. More preferably, the sulfonyl azide is a halogenated hydrocarbyloxysulfonyl azide. In one preferred embodiment, the aziridine is prepared by a highly diastereo- and enantio-selective Co-catalyzed asymmetric aziridination of alkenes with tricholoroethoxysulfonyl azide (TcesN₃).

In accordance with the process of the present invention, compounds containing an ethylenic bond, commonly known as alkenes or olefins, are aziridinated with an alkoxysulfonyl azide reagent in the presence of a metal porphyrin complex, preferably a cobalt porphyrin complex. In one preferred embodiment, the reaction mixture comprises a catalytic amount of Pd(II) such as paladium diacetate. Advantageously, the catalytic system of the present invention is operationally simple and capable of aziridinating both aromatic and aliphatic alkenes under mild conditions, forming the corresponding aziridines in high yields and excellent enantioselectivities.

Alkenes

In general, the alkene may be any of a wide range of alkenes. In one embodiment, the alkene corresponds to Formula 1:

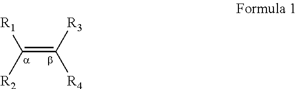

wherein R₁ and R₂ are substituents of the α-carbon of the ethylenic bond, and R₃ and R₄ are substituents of the β-carbon of the ethylenic bond. Preferably, R₁, R₂, R₃ and R₄ are independently hydrogen, hydrocarbyl, substituted hydrocarbyl, heterocyclo or EWG (electron-withdrawing group). In one embodiment, R₁, R₂, R₃ and R₄ are independently hydrogen, hydrocarbyl, substituted hydrocarbyl, or heterocyclo. In one embodiment, R₁ is hydrogen. In another embodiment, R₁ is alkyl or substituted alkyl. In one embodiment, R₂ is hydrogen. In another embodiment, R₂ is alkyl or substituted alkyl. In one embodiment, R₂ is acyl. For example, R₂ may be —C(O)R, —C(O)OR, or —C(O)NRₐR_{b} wherein R, Rₐ, and R_{b} are independently optionally substituted alkyl or optionally substituted aryl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, at least one of R₁, R₂, R₃ and R₄ is hydrogen and the other three are alkyl, substituted alkyl, aryl or substituted aryl. In one embodiment, at least two of R₁, R₂, R₃ and R₄ are hydrogen and the other two are alkyl or substituted alkyl. In one embodiment, at least two of R₁, R₂, R₃ and R₄ are hydrogen and the other two are alkyl, substituted alkyl, aryl or substituted aryl. In another embodiment, at least three of R₁, R₂, R₃ and R₄ are hydrogen and the other one is alkyl or substituted alkyl. In one embodiment, R₁, R₂ and the α-carbon, or R₃, R₄ and the β-carbon form a carbocyclic or heterocyclic ring. In another embodiment, R₁, R₃, the α-carbon, and the β-carbon or R₂, R₄, the α-carbon, and the β-carbon form a carbocyclic or heterocyclic ring. In another embodiment, R₁, R₄, the α-carbon, and the β-carbon or R₂, R₃, the α-carbon, and the β-carbon form a carbocyclic or heterocyclic ring.

In one embodiment, the alkene corresponds to Formula 1, and one of R₁, R₂, R₃ or R₄ is hydrogen. Alkenes with this substitution pattern correspond to Formulas 2a, 2b, 2c, or 2d, respectively:

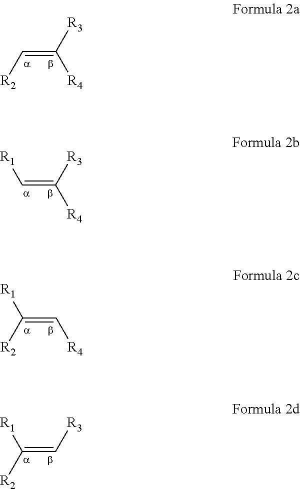

wherein R₁ and R₂ are substituents of the α-carbon of the ethylenic bond, and R₃ and R₄ are substituents of the β-carbon of the ethylenic bond. Preferably, R₁, R₂, R₃ and R₄ are independently hydrocarbyl, substituted hydrocarbyl, heterocyclo or EWG (electron-withdrawing group), provided that one of R₁, R₂, R₃ and R₄ is hydrogen. In one embodiment, R₁, R₂, R₃ and R₄ are independently hydrogen, hydrocarbyl, substituted hydrocarbyl, or heterocyclo, provided that one of R₁, R₂, R₃ and R₄ is hydrogen. In one embodiment, R₁ is hydrogen. In another embodiment, R₁ is alkyl or substituted alkyl. In one embodiment, R₂ is hydrogen. In another embodiment, R₂ is alkyl or substituted alkyl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, R₁, R₂ and the α-carbon, or R₃, R₄ and the β-carbon form a carbocyclic or heterocyclic ring. In another embodiment, R₁, R₃, the α-carbon, and the β-carbon or R₂, R₄, the α-carbon, and the β-carbon form a carbocyclic or heterocyclic ring. In another embodiment, R₁, R₄, the α-carbon, and the β-carbon or R₂, R₃, the α-carbon, and the β-carbon form a carbocyclic or heterocyclic ring.

For example, in one embodiment, the alkene corresponds to Formula 2a, 2b, 2c, or 2d and R₁, R₂, R₃, and R₄ are independently hydrogen, hydrocarbyl, substituted hydrocarbyl, heterocyclo, or an electron withdrawing group. In one embodiment, R₁ is hydrogen. In one embodiment, R₂ is hydrogen. In another embodiment, R₂ is alkyl or substituted alkyl. In another embodiment, R₂ is aryl or substituted aryl; for example, R₂ may be phenyl or substituted phenyl wherein the phenyl substituents are selected from the group consisting of alkyl, alkoxy, halo, trihalomethyl, acyloxy, and nitro. In one embodiment, R₂ is acyl; for example, R₂ may be —C(O)R, —C(O)OR, or —C(O)NRₐR_{b} wherein R, Rₐ, and R_{b} are independently optionally substituted alkyl or optionally substituted aryl. In one embodiment, R₃ is hydrogen. In another embodiment, R₃ is alkyl or substituted alkyl. In another embodiment, R₃ is aryl or substituted aryl; for example, R₃ may be phenyl or substituted phenyl wherein the phenyl substituents are selected from the group consisting of alkyl, alkoxy, halo, trihalomethyl, acyloxy, and nitro. In one embodiment, R₄ is hydrogen. In another embodiment, R₄ is alkyl or substituted alkyl. In another embodiment, R₄ is aryl or substituted aryl; for example, R₄ may be phenyl or substituted phenyl wherein the phenyl substituents are selected from the group consisting of alkyl, alkoxy, halo, trihalomethyl, acyloxy, and nitro. In one embodiment, R₃ and R₄ are both hydrogen. In one embodiment, one of R₂, R₃, and R₄ is an electron withdrawing group. In one embodiment, R₂ is an electron withdrawing group selected from the group consisting of X, —C(O)H, —C(O)R, —C(O)OR, —C(O)OH, —C(O)X, —C(X)₃, —ON, —SO₃H, —N⁺H₃, —N⁺R₃, or —N⁺O₂ where R is hydrocarbyl, substituted hydrocarbyl or heterocyclo and X is halogen, optionally substituted alkyl or optionally substituted aryl. In one embodiment, R₃, R₄ and the α-carbon form a carbocyclic or heterocyclic ring. In another embodiment, R₂, R₄, the α-carbon, and the β-carbon form a carbocyclic or heterocyclic ring. In another embodiment, R₂, R₃, the α-carbon, and the β-carbon form a carbocyclic or heterocyclic ring.

In one preferred embodiment, the alkene corresponds to Formula 1, R₄ is hydrogen, and one of R₁, R₂ and R₃ is hydrogen. Alkenes having this substitution pattern are depicted by Formula 3a, Formula 3b and Formula 3c:

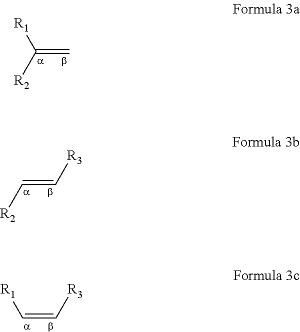

wherein R₁, R₂, and R₃ are independently hydrocarbyl, substituted hydrocarbyl, heterocyclo or EWG (electron-withdrawing group). In one embodiment, R₁, R₂, and R₃ are independently hydrogen, hydrocarbyl, substituted hydrocarbyl, or heterocyclo. In one embodiment, R₁, R₂ and the α-carbon form a carbocyclic or heterocyclic ring. In another embodiment, R₁, R₃, the α-carbon and the β-carbon form a carbocyclic or heterocyclic ring. In another embodiment, R₂, R₃, the α-carbon and the β-carbon form a carbocyclic or heterocyclic ring.

For example, in one embodiment, the alkene corresponds to Formula 3a, 3b, or 3c and R₁, R₂ and R₃ are independently hydrogen, hydrocarbyl, substituted hydrocarbyl, heterocyclo, or an electron withdrawing group. In one embodiment, R₁ is hydrogen. In another embodiment, R₁ is alkyl or substituted alkyl. In another embodiment, R₁ is aryl or substituted aryl; for example, R₁ may be phenyl or substituted phenyl wherein the phenyl substituents are selected from the group consisting of alkyl, alkoxy, halo, trihalomethyl, acyloxy, and nitro. In one embodiment, R₁ is acyl; for example, R₁ may be —C(O)R, —C(O)OR, or —C(O)NRₐR_{b} wherein R, Rₐ, and R_{b} are independently optionally substituted alkyl or optionally substituted aryl. In one embodiment, R₂ is hydrogen. In another embodiment, R₂ is alkyl or substituted alkyl. In another embodiment, R₂ is aryl or substituted aryl; for example, R₂ may be phenyl or substituted phenyl wherein the phenyl substituents are selected from the group consisting of alkyl, alkoxy, halo, trihalomethyl, acyloxy, and nitro. In one embodiment, R₂ is acyl; for example, R₂ may be —C(O)R, —C(O)OR, or —C(O)NRₐR_{b} wherein R, Rₐ, and R_{b} are independently optionally substituted alkyl or optionally substituted aryl. In one embodiment, R₃ is hydrogen. In another embodiment, R₃ is alkyl or substituted alkyl. In another embodiment, R₃ is aryl or substituted aryl; for example, R₃ may be phenyl or substituted phenyl wherein the phenyl substituents are selected from the group consisting of alkyl, alkoxy, halo, trihalomethyl, acyloxy, and nitro. In one embodiment, R₃ is acyl; for example, R₃ may be —C(O)R, —C(O)OR, or —C(O)NRₐR_{b} wherein R, Rₐ and R_{b} are independently optionally substituted alkyl or optionally substituted aryl. In one embodiment, one of R₁ and R₂ is an electron withdrawing group. In one embodiment, one of R₁ and R₃ is an electron withdrawing group. In one embodiment, R₃ is an electron withdrawing group selected from the group consisting of X, —C(O)H, —C(O)R, —C(O)OR, —C(O)OH, —C(O)X, —C(X)₃, —CN, —SO₃H, —N⁺H₃, —N⁺R₃, or —N⁺O₂ where R is hydrocarbyl, substituted hydrocarbyl or heterocyclo and X is halogen, optionally substituted alkyl or optionally substituted aryl. In one embodiment, R₁, R₂, and the α-carbon form a carbocyclic or heterocyclic ring. In another embodiment, R₁, R₃, the α-carbon, and the β-carbon form a carbocyclic or heterocyclic ring or R₂, R₃, the α-carbon, and the β-carbon form a carbocyclic or heterocyclic ring.

In another embodiment, the olefin corresponds to Formula 1 and R₂, R₃ and R₄ are hydrogen. Olefins having this substitution pattern are depicted by Formula 4:

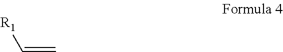

wherein R₁ is hydrocarbyl, substituted hydrocarbyl, heterocyclo or EWG (electron-withdrawing group). In one embodiment, R₁ is hydrogen, hydrocarbyl, substituted hydrocarbyl, or heterocyclo.

For example, in one such embodiment, the alkene corresponds to Formula 4, and R₁ is alkyl, substituted alkyl, aryl, substituted aryl, or acyl. In one such embodiment, R₁ is alkyl or substituted alkyl. In another such embodiment, R₁ is aryl or substituted aryl; for example, R₁ may be phenyl or substituted phenyl wherein the phenyl substituents are selected from the group consisting of alkyl, alkoxy, halo, trihalomethyl, acyloxy, and nitro. In one embodiment, R₁ is acyl; for example, R₁ may be —C(O)R, —C(O)OR, or —C(O)NRₐR_{b} wherein R, Rₐ and R_{b} are independently optionally substituted alkyl or optionally substituted aryl. In another embodiment, R₁ is alkyl. In another embodiment, R₁ is an electron withdrawing group. In another embodiment, R₁ is R₁₁C(O)— wherein R₁₁ is alkyl, substituted alkyl, alkoxy, or amino.

In general, the electron withdrawing groups, EWG, as described in connection with Formula 1, 2a, 2b, 2c, 2d, 3a, 3b, 3c, and 4 are any substituent that draws electrons away from the ethylenic bond. Exemplary electron withdrawing groups include hydroxy, alkoxy, mercapto, halogens, carbonyls, sulfonyls, nitrile, quaternary amines, nitro, trihalomethyl, imine, amidine, oxime, thioketone, thioester, or thioamide. In one embodiment, the electron withdrawing group(s) is/are hydroxy, alkoxy, mercapto, halogen, carbonyl, sulfonyl, nitrile, quaternary amine, nitro, or trihalomethyl. In another embodiment, the electron withdrawing group(s) is/are halogen, carbonyl, nitrile, quaternary amine, nitro, or trihalomethyl. In another embodiment, the electron withdrawing group(s) is/are halogen, carbonyl, nitrile, nitro, or trihalomethyl. When the electron withdrawing group is alkoxy, it generally corresponds to the formula —OR where R is hydrocarbyl, substituted hydrocarbyl, or heterocyclo. When the electron withdrawing group is mercapto, it generally corresponds to the formula —SR where R is hydrogen, hydrocarbyl, substituted hydrocarbyl or heterocyclo. When the electron withdrawing group is a halogen atom, the electron withdrawing group may be fluoro, chloro, bromo, or iodo; typically, it will be fluoro or chloro. When the electron withdrawing group is a carbonyl, it may be an aldehyde (—C(O)H), ketone (—C(O)R), ester (—C(O)OR), acid (—C(O)OH), acid halide (—C(O)X), amide (—C(O)NRₐR_{b}), or anhydride (—C(O)OC(O)R) where R is hydrocarbyl, substituted hydrocarbyl or heterocyclo, Rₐ and R_{b} are independently hydrogen, hydrocarbyl, substituted hydrocarbyl or heterocyclo, and X is a halogen atom. When the electron withdrawing group is a sulfonyl, it may be an acid (—SO₃H) or a derivative thereof (—SO₂R) where R is hydrocarbyl, substituted hydrocarbyl or heterocyclo. When the electron withdrawing group is a quaternary amine, it generally corresponds to the formula —N⁺RₐR_{b}R_{c} where Rₐ, R_{b} and R_{c} are independently hydrogen, hydrocarbyl, substituted hydrocarbyl or heterocyclo. When the electron withdrawing group is a trihalomethyl, it is preferably trifluoromethyl or trichloromethyl. In each of the foregoing exemplary electron withdrawing groups containing the variable “X”, in one embodiment, X may be chloro or fluoro, preferably fluoro. In each of the foregoing exemplary electron withdrawing groups containing the variable “R”, R may be alkyl. In each of the foregoing exemplary electron withdrawing groups containing the variable “Rₐ” and “R_{b}”, Rₐ and R_{b} may independently be hydrogen or alkyl.

In accordance with one preferred embodiment, the electron withdrawing group(s) is/are a halide, aldehyde, ketone, ester, carboxylic acid, amide, acyl chloride, trifluoromethyl, nitrile, sulfonic acid, ammonia, amine, or a nitro group. In this embodiment, the electron withdrawing group(s) correspond to one of the following chemical structures: —X, —C(O)H, —C(O)R, —C(O)OR, —C(O)OH, —C(O)X, —C(X)₃, —CN, —SO₃H, —N⁺H₃, —N⁺R₃, or —N⁺O₂ where R is hydrocarbyl, substituted hydrocarbyl or heterocyclo and X is halogen.

Nitrene Source

In general, the olefin is aziridinated with a nitrene source. Preferably, the nitrene source is an azide reagent (also sometimes referred to herein as an azide compound) wherein the nitrene is generated by the removal of N₂ as nitrogen gas from the solution. More preferably, the nitrene precursor is a sulfonyl azide. In one embodiment, the azide compound corresponds to the following structure:

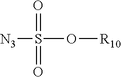

wherein R₁₀ is hydrogen, hydrocarbyl, substituted hydrocarbyl, or heterocyclo. In one embodiment, R₁₀ is alkyl, substituted alkyl, alkenyl, alkynyl, phenyl, or heterosubstituted phenyl. For example, in one embodiment, R₁₀ is halogenated alkyl such as trihalomethyl, trihaloethyl, etc. In one embodiment, R₁₀ is trichloroalkyl such as trichloromethyl or trichloroethyl. More preferably, the nitrene precursor is trichloroethoxysulfonyl azide, also referred to herein as TcesN₃:

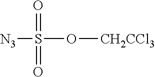

Aziridination

In accordance with one embodiment of the present invention, an alkene is converted to an aziridine as illustrated in Reaction Scheme A:

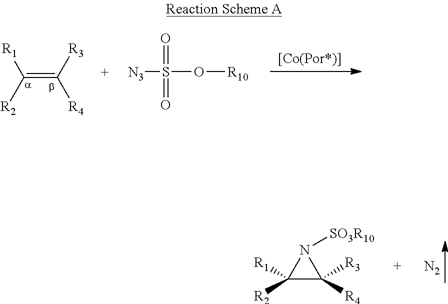

wherein [Co(Por*)] is a cobalt porphyrin complex, R₁, R₂, R₃ and R₄ are as defined in connection with any of Formulae 1, 2a, 2b, 2c, 2d, 3a, 3b, 3c, or 4, and R₁₀ is hydrogen, hydrocarbyl, substituted hydrocarbyl, or heterocyclo. Thus, for example, in one embodiment, R₁, R₂, R₃ and R₄ are independently hydrogen, hydrocarbyl, substituted hydrocarbyl, heterocyclo, or electron withdrawing group. In one embodiment, R₁, R₂, R₃ and R₄ are independently hydrogen, hydrocarbyl, substituted hydrocarbyl, or heterocyclo, and R₁₀ is hydrogen, hydrocarbyl, substituted hydrocarbyl, or heterocyclo. In one embodiment, R₁₀ is alkyl, substituted alkyl, alkenyl, alkynyl, phenyl, or heterosubstituted phenyl. In another embodiment, R₁₀ is trichloroethyl. In one embodiment, R₁ is hydrogen. In another embodiment, R₁ is alkyl or substituted alkyl. In one embodiment, R₂ is hydrogen. In another embodiment, R₂ is alkyl or substituted alkyl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, at least one of R₁, R₂, R₃ and R₄ is hydrogen and the other three are alkyl or substituted alkyl. In one embodiment, at least two of R₁, R₂, R₃ and R₄ are hydrogen and the other two are alkyl or substituted alkyl. In another embodiment, at least three of R₁, R₂, R₃ and R₄ are hydrogen and the other one is alkyl or substituted alkyl. In one embodiment, R₁, R₂ and the aziridine ring carbon to which they are bonded, or R₃, R₄ and the aziridine ring carbon to which they are bonded, form a carbocyclic or heterocyclic ring. In another embodiment, R₁, R₃, and the aziridine ring carbons to which R₁ and R₃ are bonded, or R₂, R₄, and the aziridine ring carbons to which R₂ and R₄ are bonded, form a carbocyclic or heterocyclic ring. In another embodiment, R₁, R₄, and the aziridine ring carbons to which R₁ and R₄ are bonded, or R₂, R₃, and the aziridine ring carbons to which R₂ and R₃ are bonded, form a carbocyclic or heterocyclic ring.

In one preferred embodiment, an alkene is converted to an aziridine as illustrated in Reaction Scheme B-1, B-2, B-3, or B-4.

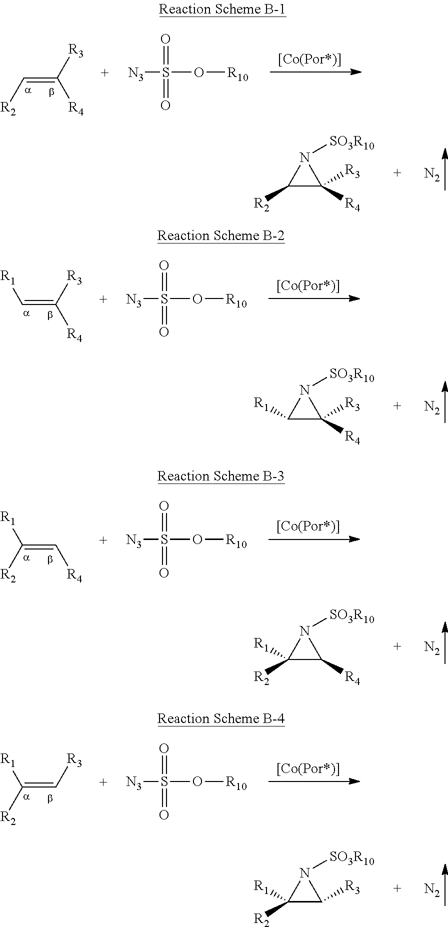

wherein [Co(Por*)] is a cobalt porphyrin complex, R₁, R₂, R₃ and R₄ are independently hydrogen, hydrocarbyl, substituted hydrocarbyl, heterocyclo, or electron withdrawing group and R₁₀ is hydrogen, hydrocarbyl, substituted hydrocarbyl, or heterocyclo. In one embodiment, R₁, R₂, R₃ and R₄ are independently hydrogen, hydrocarbyl, substituted hydrocarbyl, or heterocyclo, and R₁₀ is hydrogen, hydrocarbyl, substituted hydrocarbyl, or heterocyclo. In one embodiment, R₁₀ is alkyl, substituted alkyl, alkenyl, alkynyl, phenyl, or heterosubstituted phenyl. In another embodiment, R₁₀ is trichloroethyl. In one embodiment, R₁ is hydrogen. In another embodiment, R₁ is alkyl or substituted alkyl. In one embodiment, R₂ is hydrogen. In another embodiment, R₂ is alkyl or substituted alkyl. In one embodiment, R₃ is hydrogen. In another embodiment, R₃ is alkyl or substituted alkyl. In one embodiment, R₄ is hydrogen. In another embodiment, R₄ is alkyl or substituted alkyl. In one embodiment, at least one of R₁, R₂, R₃ and R₄ is hydrogen and the other two are independently alkyl or substituted alkyl. In one embodiment, at least two of R₁, R₂, R₃ and R₄ are hydrogen and the other one is alkyl or substituted alkyl. In one embodiment, R₁, R₂ and the aziridine ring carbon to which they are bonded, or R₃, R₄ and the aziridine ring carbon to which they are bonded, form a carbocyclic or heterocyclic ring. In another embodiment, R₁, R₃, and the aziridine ring carbons to which R₁ and R₃ are bonded, or R₂, R₄, and the aziridine ring carbons to which R₂ and R₄ are bonded, form a carbocyclic or heterocyclic ring. In another embodiment, R₁, R₄, and the aziridine ring carbons to which R₁ and R₄ are bonded, or R₂, R₃, and the aziridine ring carbons to which R₂ and R₃ are bonded, form a carbocyclic or heterocyclic ring.

In one preferred embodiment, an alkene is converted to an aziridine as illustrated in Reaction Scheme C-1:

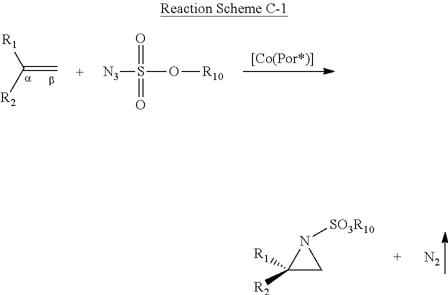

wherein [Co(Por*)] is a cobalt porphyrin complex, R₁ and R₂ are independently hydrogen, hydrocarbyl, substituted hydrocarbyl, or heterocyclo, and R₁₀ is hydrogen, hydrocarbyl, substituted hydrocarbyl, or heterocyclo. In one embodiment, R₁₀ is alkyl, substituted alkyl, alkenyl, alkynyl, phenyl, or heterosubstituted phenyl. In another embodiment, R₁₀ is trichloroethyl. In one embodiment, R₁ is hydrogen. In another embodiment, R₁ is alkyl or substituted alkyl. In one embodiment, R₂ is hydrogen. In another embodiment, R₂ is alkyl or substituted alkyl. In one embodiment, one of R₁ and R₂ is hydrogen and the other one is alkyl or substituted alkyl. In one embodiment, one of R₁ and R₂ is alkyl or substituted alkyl and the other is aryl or substituted aryl. In one embodiment, R₁ and R₂ are independently alkyl, substituted alkyl, aryl or substituted aryl. In one embodiment, R₁, R₂ and the aziridine ring carbons to which they are bonded form a carbocyclic or heterocyclic ring.

In another preferred embodiment, an alkene is converted to an aziridine as illustrated in Reaction Scheme C-2:

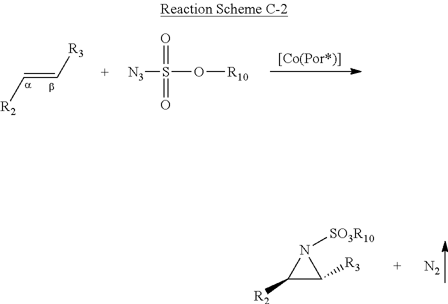

wherein [Co(Por*)] is a cobalt porphyrin complex, R₂ and R₃ are independently hydrogen, hydrocarbyl, substituted hydrocarbyl, or heterocyclo, and R₁₀ is hydrogen, hydrocarbyl, substituted hydrocarbyl, or heterocyclo. In one embodiment, R₁₀ is alkyl, substituted alkyl, alkenyl, alkynyl, phenyl, or heterosubstituted phenyl. In another embodiment, R₁₀ is trichloroethyl. In one embodiment, R₂ is hydrogen. In another embodiment, R₂ is alkyl or substituted alkyl. In one embodiment, R₃ is hydrogen. In another embodiment, R₃ is alkyl or substituted alkyl. In one embodiment, one of R₂ and R₃ is hydrogen and the other one is alkyl or substituted alkyl. In one embodiment, one of R₂ and R₃ is alkyl or substituted alkyl and the other is aryl or substituted aryl. In one embodiment, R₂ and R₃ are independently alkyl, substituted alkyl, aryl or substituted aryl. In one embodiment, R₂, R₃, and the aziridine ring carbons to which they are bonded form a carbocyclic or heterocyclic ring.

In another preferred embodiment, an alkene is converted to an aziridine as illustrated in Reaction Scheme C-3:

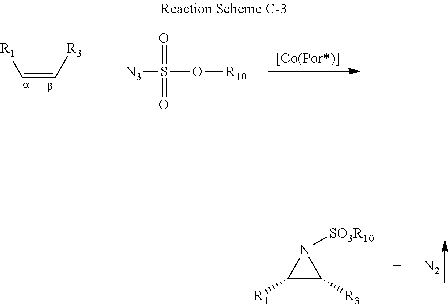

wherein [Co(Por*)] is a cobalt porphyrin complex, R₁ and R₃ are independently hydrogen, hydrocarbyl, substituted hydrocarbyl, or heterocyclo, and R₁₀ is hydrogen, hydrocarbyl, substituted hydrocarbyl, or heterocyclo. In one embodiment, R₁₀ is alkyl, substituted alkyl, alkenyl, alkynyl, phenyl, or heterosubstituted phenyl. In another embodiment, R₁₀ is trichloroethyl. In one embodiment, R₁ is hydrogen. In another embodiment, R₁ is alkyl or substituted alkyl. In one embodiment, R₃ is hydrogen. In another embodiment, R₃ is alkyl or substituted alkyl. In one embodiment, one of R₁ and R₃ is hydrogen and the other one is alkyl or substituted alkyl. In one embodiment, one of R₁ and R₃ is alkyl or substituted alkyl and the other is aryl or substituted aryl. In one embodiment, R₁ and R₃ are independently alkyl, substituted alkyl, aryl or substituted aryl. In one embodiment, R₁, R₃, and the aziridine ring carbons to which R₁ and R₃ are bonded form a carbocyclic or heterocyclic ring.

In another preferred embodiment, an alkene is converted to an aziridine as illustrated in Reaction Scheme D:

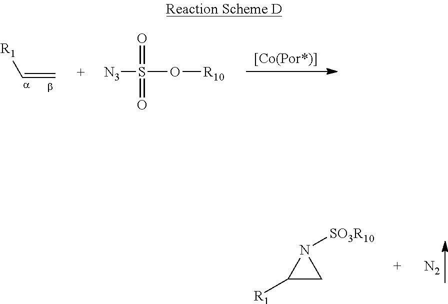

wherein [Co(Por*)] is a cobalt porphyrin complex, R₁ is hydrogen, hydrocarbyl, substituted hydrocarbyl, or heterocyclo, and R₁₀ is hydrogen, hydrocarbyl, substituted hydrocarbyl, or heterocyclo. In one embodiment, R₁₀ is alkyl, substituted alkyl, alkenyl, alkynyl, phenyl, or heterosubstituted phenyl. In another embodiment, R₁₀ is trichloroethane. In one embodiment, R₁ is hydrogen. In another embodiment, R₁ is alkyl or substituted alkyl.

In a further preferred embodiment, an alkene is converted to an aziridine as illustrated in Reaction Scheme E:

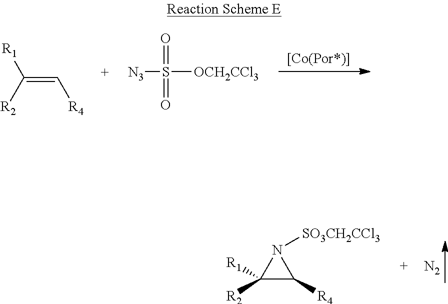

wherein [Co(Por*)] is a cobalt porphyrin complex and R₁, R₂ and R₄ are independently hydrogen, hydrocarbyl, substituted hydrocarbyl, or heterocyclo. In this embodiment, the nitrene source is trichloroethoxysulfonyl azide. In one embodiment, R₁ is hydrogen. In another embodiment, R₁ is alkyl or substituted alkyl. In one embodiment, R₂ is hydrogen. In another embodiment, R₂ is alkyl or substituted alkyl. In one embodiment, R₄ is hydrogen. In another embodiment, R₄ is alkyl or substituted alkyl. In one embodiment, one of R₁, R₂ and R₄ is hydrogen and the other two are independently alkyl or substituted alkyl. In one embodiment, at least two of R₁, R₂ and R₄ are hydrogen and the other one is alkyl or substituted alkyl. In one embodiment, one of R₁, R₂ and R₄ is hydrogen and the other two are independently alkyl, substituted alkyl, aryl or substituted aryl. In one embodiment, R₁, R₂ and the aziridine ring carbon to which they are bonded form a carbocyclic or heterocyclic ring. In another embodiment, R₁, R₄, and the aziridine ring carbons to which R₁ and R₃ are bonded form a carbocyclic or heterocyclic ring. In another embodiment, R₂, R₄, and the aziridine ring carbons to which R₂ and R₄ are bonded form a carbocyclic or heterocyclic ring. In a preferred embodiment, [Co(Por*)] is [Co(P6)] as described below.

The enantioselectivity and diastereoselectivity can be influenced, at least in part, by selection of the cobalt porphyrin complex. Similarly, stereoselectivity of the reaction may also be influenced by the selection of chiral porphyrin ligands with desired electronic, steric, and chiral environments. Accordingly, the catalytic system of the present invention may advantageously be used to control stereoselectivity.

In each of the foregoing embodiments in which the aziridine corresponds to the aziridine product depicted in any of Reaction Schemes A, B-1, B-2, B-3, B-4, C-1, C-2, C-3, D or E, it is generally preferred that the enanatiomeric excess of the aziridine stereoisomer over its enantiomer in the reaction product mixture be at least 60%. More preferably, the enanatiomeric excess of the aziridine product depicted in any of Reaction Schemes A, B-1, B-2, B-3, B-4, C-1, C-2, C-3, D or E over its enantiomer be at least at least 70%. More preferably, the enanatiomeric excess of the aziridine product depicted in any of Reaction Schemes A, B-1, B-2, B-3, B-4, C-1, C-2, C-3, D or E over its enantiomer be at least at least 80%. Still more preferably, the enanatiomeric excess of the aziridine product depicted in any of Reaction Schemes A, B-1, B-2, B-3, B-4, C-1, C-2, C-3, D or E over its enantiomer be at least 90%. In certain embodiments, the enanatiomeric excess of the aziridine product depicted in any of Reaction Schemes A, B-1, B-2, B-3, B-4, C-1, C-2, C-3, D or E over its enantiomer is at least 95%.

In any of any of the foregoing embodiments in which the aziridine product depicted in any of Reaction Schemes A, B-1, B-2, B-3, B-4, C-1, C-2, C-3, D or E contains a mixture of diastereomers, it is generally preferred that the ratio of the desired aziridine stereoisomer to its diastereomer be greater than 90:1, more preferably greater than 98:1 and still more preferably at least 99:1, respectively.

Metal Porphyrins

In one embodiment, the metal of the metal porphyrin complex is a transition metal. Thus, for example, the metal may be any of the 30 metals in the 3d, 4d, and 5d transition metal series of the Periodic Table of the Elements, including the 3d series that includes Sc, Ti, V, Cr, Mn, Fe, Co, Ni, Cu, and Zn; the 4d series that includes Y, Zr, Nb, Mo, Tc, Ru, Rh, Pd, Ag and Cd; and the 5d series that includes Lu, Hf, Ta, W, Re, Os, Ir, Pt, Au and Hg. In some embodiments, M is a transition metal from the 3d series. In some embodiments, M is selected from the group consisting of Co, Zn, Fe, Ru, Mn, and Ni. In some embodiments, M is selected from the group consisting of Co, Fe, and Ru. In some embodiments, M is Co.

The porphyrin with which cobalt is complexed may be any of a wide range of porphyrins known in the art. Exemplary porphyrins are described in U.S. Patent Publication Nos. 2005/0124596 and 2006/0030718 and U.S. Pat. No. 6,951,934 (each of which is incorporated herein by reference, in its entirety). Exemplary porphyrins are also described in Chen et al., Bromoporphyrins as Versatile Synthons for Modular Construction of Chiral Porphyrins: Cobalt-Catalyzed Highly Enantioselective and Diastereoselective Cyclopropanation (J. Am. Chem. Soc. 2004), which is incorporated herein by reference in its entirety. D₂-symmetrical chiral porphyrins can be effectively synthesized from bromoporphyrins via Pd-mediated quadruple amidation: see, e.g., Chen et al., J. Am. Chem. Soc. 2004, 126, 14718; and Zhu et al., J. Am. Chem. Soc. 2008, 130, 5042. See, also, Doyle, Angew. Chem. Int. Ed., 2009, 48, 850.

In one embodiment, the cobalt porphyrin complex is a cobalt (II) porphyrin complex. In one particularly preferred embodiment, the cobalt porphyrin complex is a D₂-symmetric chiral porphyrin complex corresponding to the following structure

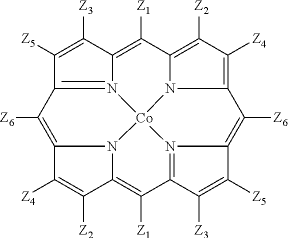

wherein each Z₁, Z₂, Z₃, Z₄, Z₅ and Z₆ are each independently selected from the group consisting of X, H, alkyl, substituted alkyls, arylalkyls, aryls and substituted aryls; and X is selected from the group consisting of halogen, trifluoromethanesulfonate (OTf), haloaryl and haloalkyl. In a preferred embodiment, Z₂, Z₃, Z₄ and Z₅ are hydrogen, Z₁ is a substituted phenyl, and Z₆ is substituted phenyl, and Z₁ and Z₆ are different. In one particularly preferred embodiment, Z₂, Z₃, Z₄ and Z₅ are hydrogen, Z₁ is substituted phenyl, and Z₆ is substituted phenyl and Z₁ and Z₆ are different and the porphyrin is a chiral porphyrin. In one even further preferred embodiment, Z₂, Z₃, Z₄ and Z₅ are hydrogen, Z₁ is substituted phenyl, and Z₆ is substituted phenyl and Z₁ and Z₆ are different and the porphyrin has D₂-symmetry.

In one embodiment, Z₁ is selected from the group consisting of

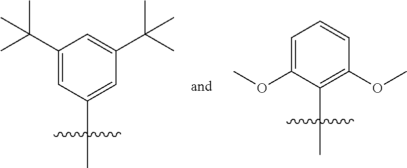

wherein

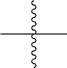

denotes the point of attachment to the porphyrin complex.

In one embodiment, Z₆ is selected from the group consisting of

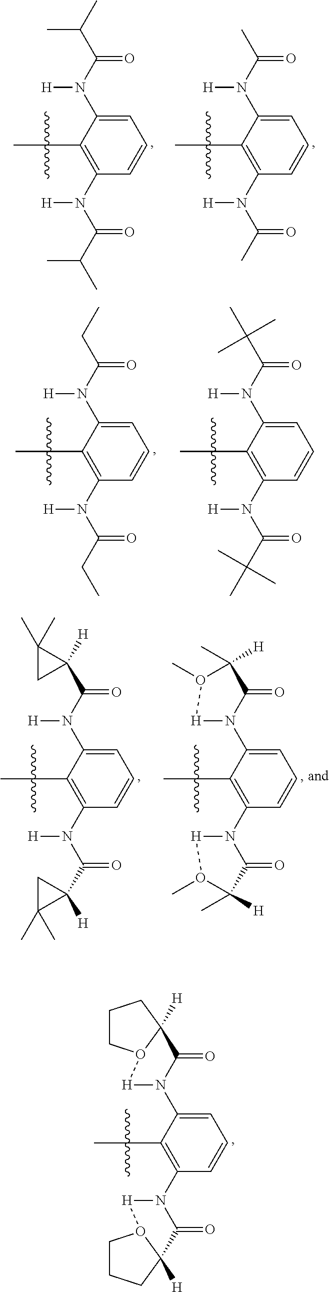

wherein

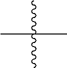

denotes the point of attachment to the porphyrin complex.

Exemplary cobalt (II) porphyrins having D₂-symmetry for use in accordance with one aspect of the present invention include those corresponding to Formulae [Co(P1)], [Co(P2)], [Co(P3)], [Co(P4)], [Co(P5)], and [Co(P6)]:

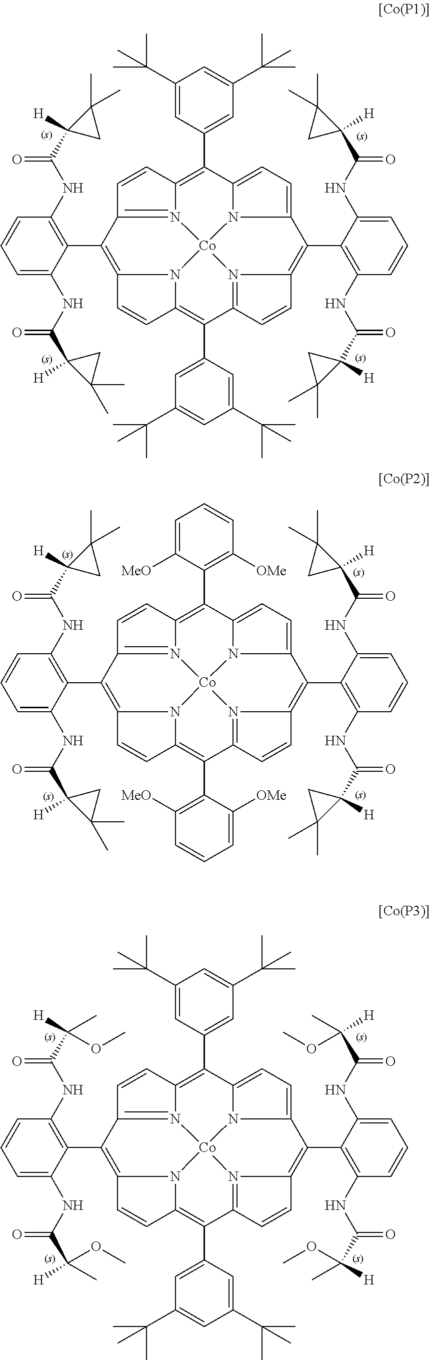

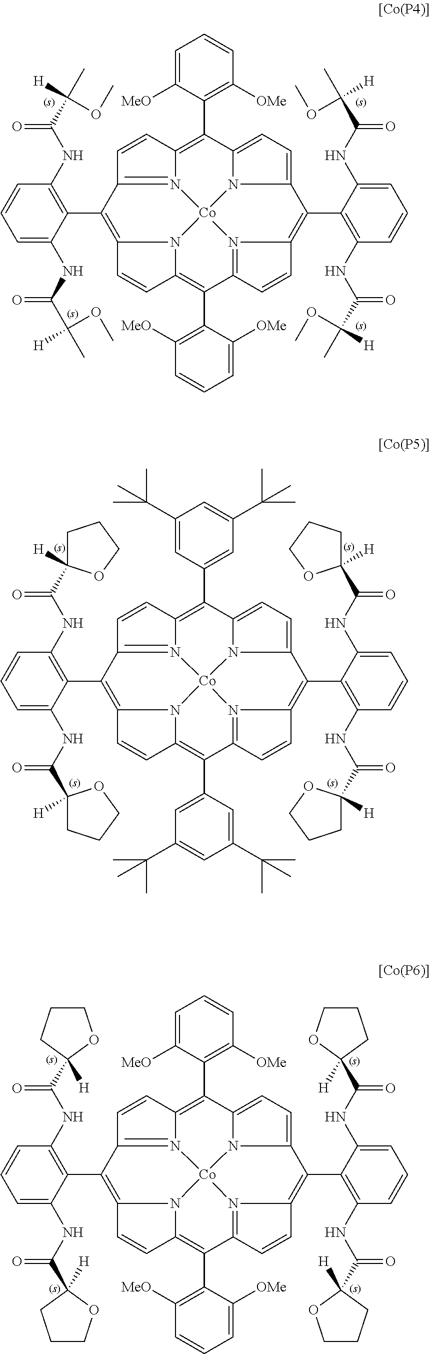

In one preferred embodiment, the cobalt porphyrin complex corresponds to [Co(P6)].

Other exemplary cobalt(II) porphyrins include the following, designated [Co(P11)], [Co(P12)], [Co(P13)], and [Co(P14)]:

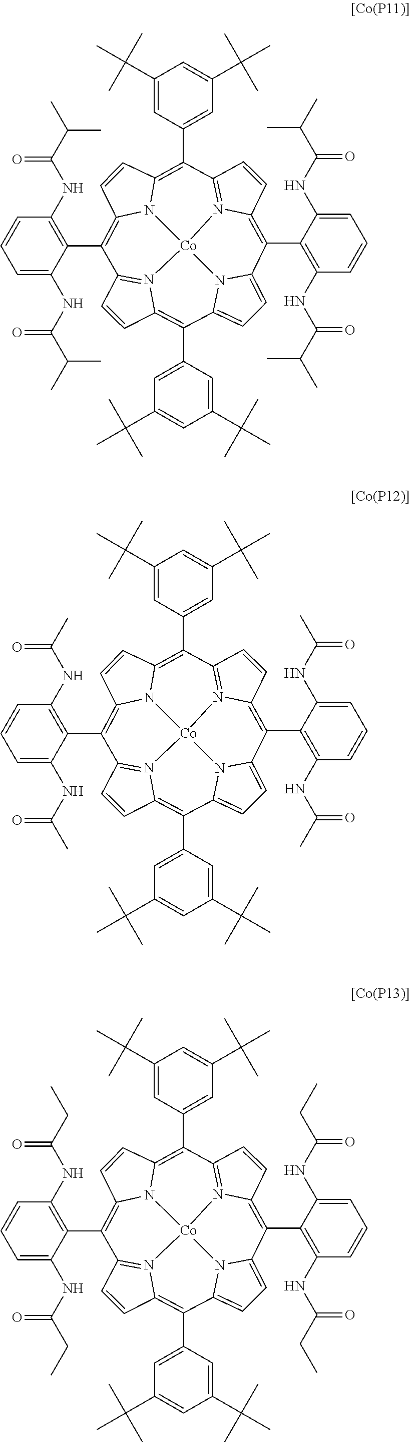

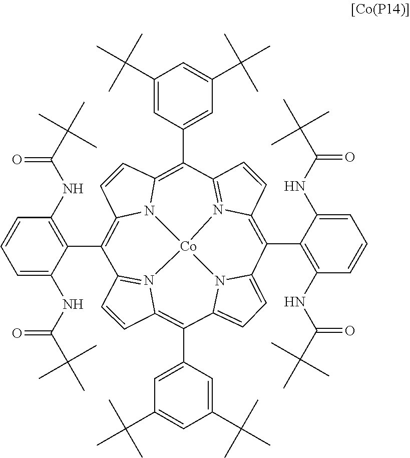

Having described the invention in detail, it will be apparent that modifications and variations are possible without departing the scope of the invention defined in the appended claims. Furthermore, it should be appreciated that all examples in the present disclosure are provided as non-limiting examples.

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### EXAMPLES

General Considerations. All cross-coupling and aziridination reactions were performed under nitrogen in oven-dried glassware following standard Schlenk techniques. 4 Å molecular sieves were dried in a vacuum oven prior to use. Chlorobenzene, acetonitrile, and dichloromethane were dried over calcium hydride under nitrogen and freshly distilled before use. Toluene and tetrahydrofuran were distilled under nitrogen from sodium benzophenone ketyl prior to use. Co-Chiral Porphyrins were prepared from reported procedure¹. Thin layer chromatography was performed on Merck TLC plates (silica gel 60 F254). Flash column chromatography was performed with ICN silica gel (60 Å, 230-400 mesh, 32-63 μm). ¹H NMR and ¹³C NMR were recorded on a Varian Inova400 (400 MHz) instrument with chemical shifts reported relative to residual solvent. Infrared spectra were measured with a Nicolet Avatar 320 spectrometer with a Smart Miracle accessory. HPLC measurements were carried out on a Shimadzu HPLC system with a Whelk-O 1, Chiralcel OD-H, or Chiralcel OJ-H column. HRMS data was obtained on an Agilent 1100 LC/MS ESI/TOF mass spectrometer with electrospray ionization. Optical rotation was measured on a Rudolf Autopol IV Polarimeter.

Synthesis of Trichloroethoxysulfonyl Azide. The 2,2,2-trichloroethanol (1.91 ml, 20 mmol) was dissolved in DCM (20 mL). Pyridine (10 mL) was added in one portion at 0° C., and the resulting solution was stirred for 15-20 minutes. Sulfuryl chloride (1.78 mL, 22 mmol in 20 mL DCM) was added dropwise over 20-30 minutes. The reaction mixture was allowed to warm up to room temperature and stirred overnight. After the reaction was complete, the flask underwent rotary evaporation until the DCM was removed. The residue was dissolved in 10 mL CH₃CN and the solution was stirred at 0° C. for 15-20 minutes. Sodium azide (1.95 g, 1.5 eq) was added in one portion to the sufuryl chloride mixture and the reaction mixture was allowed to warm up to room temperature and stirred overnight. After the reaction was complete, the flask underwent rotary evaporation until the acetonitrile was removed. The crude product was extracted from the water using ethyl acetate (3×50 mL). It was then washed with brine (20 mL), dried over sodium sulfate, and concentrated by rotary evaporation. The resulting oil was then purified by flash column chromatography. The fractions containing product were collected and concentrated by rotary evaporation to afford a colorless oily liquid (4.3 g, 84%).

The X-ray intensities were measured using Bruker-AXS SMART APEX/CCD diffractometer (MoKα, λ=0.71073 Å). Indexing was performed using SMART v5.625. Frames were integrated with SaintPlus 6.01 software package. Absorption correction was performed by multi-scan method implemented in SADABS. The structure was solved using SHELXS-97 and refined using SHELXL-97 contained in SHELXTL v6.10 and WinGX v1.70.01 program packages. All non-hydrogen atoms were refined anisotropically. Absolute configuration (and absolute structure) was established by anomalous-dispersion effects in diffraction measurements on the crystal. Crystal data and refinement conditions are shown in Table 1A.

TABLE 1A
Crystal data and structure refinement for (4-Chloro-phenyl)-aziridine-1-
sulfonic acid 2,2,2-trichloro-ethyl ester

| Empirical formula | C10H9Cl4NO3S | |
|---|---|---|
| Formula weight | 365.04 | |
| Temperature | 296(2) K | |
| Wavelength | 1.54178 Å | |
| Crystal system | Monoclinic | |
| Space group | P2(1) | |
| Unit cell dimensions | a = 10.4908(6) Å | α = 90°. |
| | b = 6.0700(4) Å | β = 104.370(3)°. |
| | c = 12.0008(7) Å | γ = 90°. |

| | |
|---|---|
| Volume | 740.29(8) Å³ |
| Z | 2 |
| Density (calculated) | 1.638 Mg/m³ |
| Absorption coefficient | 8.624 mm⁻¹ |
| F(000) | 368 |
| Crystal size | 0.40 × 0.20 × 0.10 mm³ |
| Theta range for data collection | 3.80 to 68.13°. |
| Index ranges | −12 <= h <= 12, −7 <= k <= 6, |
| | −14 <= l <= 13 |
| Reflections collected | 6397 |
| Independent reflections | 2367 [R(int) = 0.0384] |
| Completeness to theta = 68.13° | 96.6% |
| Absorption correction | Semi-empirical from equivalents |
| Max. and min. transmission | 0.4793 and 0.1299 |
| Refinement method | Full-matrix least-squares on F₂ |
| Data/restraints/parameters | 2367/1/172 |
| Goodness-of-fit on F₂ | 1.037 |
| Final R indices [I > 2sigma(I)] | R1 = 0.0347, wR2 = 0.0831 |
| R indices (all data) | R1 = 0.0376, wR2 = 0.0851 |
| Absolute structure parameter | 0.04(2) |
| Largest diff. peak and hole | 0.275 and −0.284 e · Å⁻³ |

Aziridination. Among the various azides that were evaluated, TcesN₃, a new azide that was readily synthesized from 2,2,2-trichloroethanol in high yield and on a multi-gram scale, turned out to be one of the most promising nitrene sources for [Co(Por*)]-catalyzed asymmetric aziridination due to its high reactivity and convenient usage. Careful control experiments showed TcesN₃ was stable under various conditions, but it should be noted that certain azides may be potentially explosive and should be handled with care. For the use of trichloroethylsulfamate ester in the presence of an oxidant for metal-catalyzed aziridination, see: Guthikonda et al., J. Am. Chem. Soc. 2002, 124, 13672; Keaney et al., Tetrahedron Lett. 2005, 46, 4031; Guthikonda et al., Tetrahedron 2006, 62, 11331; Li et al., J. Org. Chem. 2006, 71, 5876; and Xu et al., Org. Lett. 2008, 10, 1497. As summarized in Tables 1 and S1 for the aziridination of styrene, although the Co(II) complexes of P1 and P2 derived from chiral cyclopropanecarboxamide could effectively catalyze the reaction at room temperature, the enantioselectivities were low (Table 1, entries 1 and 2). Employment of P3 and P4, which contain chiral methoxypropionamide, improved both yield and enantioselectivity (Table 1, entries 3 and 4). While no further improvement was observed with P5, high enantioselectivity was achieved by using [Co(P6)] where the Co(II) center is surrounded by methoxy and chiral tetrahydrofurancarboxamide units, albeit in lower yield (Table 1, entries 5 and 6). The yield of [Co(P6)]-catalyzed reaction could be greatly increased by raising catalyst loading without affecting the high ee value (Table 1, entries 7 and 8). When conducted at lower temperatures, the enantioselectivity was further improved to 94-96% ee but at a sacrifice of yields (Table 1, entries 9 and 11). Interestingly, addition of a catalytic amount of Pd(OAc)₂ resulted in a dramatic increase in yields with no effect on the high enantioselectivity (Table 1, entries 10 and 12), presumably due to the activation of styrene by the π-electrophilic Lewis acid, Pd(OAc)₂. (Yamamoto Y., J. Org. Chem. 2007, 72, 7817.)

With the optimization of catalytic conditions, the substrate scope of the [Co(P6)]/TcesN₃-based system was examined (Table 2). Like styrene (Table 2, entry 1), its various derivatives were also excellent substrates, including those substituted with alkyl (Table 2, entries 2-5) and halogen (Table 2, entries 6-10) groups at different ring positions. The corresponding aziridines were produced in high yields and with excellent enantioselectivities. In the case of 2-bromostyrene, the desired product was isolated in 92% yield and 99% ee (Table 2, entry 10). Styrene derivatives containing strongly electron-withdrawing groups, such as —CF₃ and —NO₂, were suitable substrates as well (Table 2, entries 11 and 12). Furthermore, the aziridination of 2-vinyl naphthalene and α-methlystyrene were successfully performed (Table 2, entries 13-15). When an enol derivative was used as the substrate, the corresponding α-amino ketone was obtained in high yield as a result of ring-opening of the aziridine product (Table 2, entry 16). In addition to aromatic olefins, aliphatic olefins such as 1-hexene could be aziridinated in high enantioselectivities but moderate yields (Table 2, entries 17-19). In this case, [Co(P5)] proved to give a better yield than [Co(P6)]. Finally, the [Co(P6)]/TcesN₃-based system could be also applied for aziridination of aliphatic dienes and cyclic olefins (Table 2, entries 20-22). For examples of efficient aziridination of aliphatic olefins, see Guthikonda et al., J. Am. Chem. Soc. 2002, 124, 13672; Keaney et al., Tetrahedron Lett. 2005, 46, 4031; Guthikonda et al., Tetrahedron 2006, 62, 11331; Li et al., J. Org. Chem. 2006, 71, 5876; and Xu et al., Org. Lett. 2008, 10, 1497 and Catino et al., Org. Lett., 2005, 7, 2787.

The absolute configuration of the aziridination product of 4-chlorostyrene, 2-(4-chlorophenyl)-1-aziridine-sulfonic acid 2′,2′,2′-trichloroethyl ester (Table 2, entry 6), was established to be (R) by X-ray crystallographic analysis (FIG. 1). In view of the same sign of optical rotations, all the other aziridine derivatives from aromatic olefins and conjugate dienes (Table 2, entries 1-15 and 20) are expected to have the same (R) absolute configuration by analogy. It was interesting to note that the aziridine products from the reactions of aliphatic olefins exhibited the opposite sign of optical rotations (Table 2, entries 17-19).

To enhance the practicality of the [Co(P6)]/TcesN₃-based catalytic system, a simple precipitation/filtration protocol was established to allow the recycling/reusing of the catalyst. After each catalytic cycle, [Co(P6)] could be completely precipitated by adding hexanes where it absorbed uniformly to the molecular sieves (MS) preexisted in the system. The MS-absorbed [Co(P6)] was then filtered, dried under vacuum with heating, and reused. As demonstrated with the reaction of 2-bromostyrene, the catalytic system could be recycled/reused three times without significant loss of both yield and enantioselectivity (Table 3).

In summary, we have developed a highly asymmetric aziridination system based on [Co(P6)] and TcesN₃. This represents the first highly effective and enantioselective catalytic system for asymmetric aziridination of a broad range of simple olefins, without the need of additional functionalities in the substrates for secondary binding interactions. In addition to the common attributes associated with the use of azides as the nitrene sources, a simple protocol has been established to allow the recycling/reusing of the catalyst, further enhancing its potential for practical synthetic applications. This Co-catalyzed asymmetric aziridination with TcesN₃ complements the previous Cu/ArI=NTs-based systems, which are best suitable to internal olefins having additional functionalities.

TABLE 1
Enantioselective Aziridination of Styrene with Trichloroethoxysulfonyl
Azide by Chiral Cobalt(II) Porphyrins^{a}

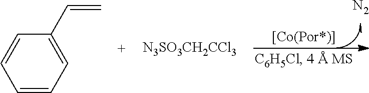

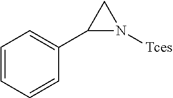

| | | | temp | yield | ee |
|---|---|---|---|---|---|
| entry | [Co(Por*)]^{b} | mol % | (° C.) | (%)^{c} | (%)^{d} |
| 1 | [Co(P1)] | 2.0 | RT | 80 | 25 |
| 2 | [Co(P2)] | 2.0 | RT | 84 | 4 |
| 3 | [Co(P3)] | 2.0 | RT | 90 | −39 |
| 4 | [Co(P4)] | 2.0 | RT | 95 | −51 |
| 5 | [Co(P5)] | 2.0 | RT | 85 | 52 |
| 6 | [Co(P6)] | 2.0 | RT | 58 | 86 |
| 7 | [Co(P6)] | 5.0 | RT | 79 | 88 |
| 8 | [Co(P6)] | 5.0 | RT | 90 | 88 |
| 9 | [Co(P6)] | 5.0 | 0 | 69 | 94 |
| 10^{e} | [Co(P6)] | 5.0 | 0 | 91 | 94 |
| 11 | [Co(P6)] | 5.0 | −10 | 15 | 96 |
| 12^{e} | [Co(P6)] | 5.0 | −10 | 59 | 96 |

^{a}Performed under N₂: styrene:TcesN₃ = 5.0:1.0; [styrene] = 0.25M.
^{b}See structures above.
^{c}Isolated yields.
^{d}Measured by chiral HPLC.
^{e}Added 5 mol % Pd(OAc)₂.

TABLE S1
Enantioselective Aziridination of Styrene with Trichloroethoxysulfonyl
Azide by Chiral Cobalt (II) Porphyrins.^{a}

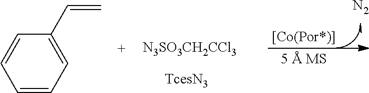

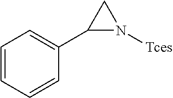

| | | | | temp | yield | ee |
|---|---|---|---|---|---|---|
| entry | [Co(Por*)]^{b} | mol % | solvent | (° C.) | (%)^{c} | (%)^{d} |
| 1 | [Co(P1)] | 2.0 | C₆H₆ | RT | 78 | 21 |
| 2 | [Co(P1)] | 2.0 | C₆H₅Cl | RT | 80 | 25 |
| 3 | [Co(P1)] | 2.0 | C₂H₄Cl₂ | RT | 88 | 25 |
| 4 | [Co(P2)] | 2.0 | C₆H₅Cl | RT | 84 | 4 |
| 5 | [Co(P3)] | 2.0 | C₆H₅Cl | RT | 90 | −39 |
| 6 | [Co(P4)] | 2.0 | C₆H₆ | RT | 78 | −40 |
| 7 | [Co(P4)] | 2.0 | C₆H₅Cl | RT | 95 | −51 |
| 8 | [Co(P4)] | 2.0 | C₂H₄Cl₂ | RT | 60 | −39 |
| 9 | [Co(P5)] | 2.0 | C₆H₅Cl | RT | 85 | 52 |
| 10 | [Co(P6)] | 2.0 | C₆H₅Cl | RT | 58 | 86 |
| 11 | [Co(P6)] | 2.0 | C₆H₅Cl | 40 | 70 | 70 |
| 12 | [Co(P6)] | 3.0 | C₆H₅Cl | RT | 79 | 88 |
| 13 | [Co(P6)] | 3.0 | C₂H₄Cl₂ | 40 | 56 | 66 |
| 14 | [Co(P6)] | 5.0 | C₆H₅Cl | RT | 90 | 88 |
| 15 | [Co(P6)] | 5.0 | C₆H₅Cl | 40 | 84 | 76 |
| 16 | [Co(P6)] | 5.0 | C₆H₅Cl | 0 | 69 | 94 |
| 17 | [Co(P6)] | 5.0 | C₆H₅Cl | 0 | 91 | 94 |
| 18 | [Co(P6)] | 5.0 | C₆H₅Cl | −10 | 15 | 96 |
| 19 | [Co(P6)] | 5.0 | C₆H₅Cl | −10 | 69 | 96 |

^{a}Performed in chlorobenzene for 24 h under N₂ in the presence of 4 Å molecular sieves: alkene:TcesN₃ = 5.0:1.0; [alkene] = 0.25M.
^{b}See structures above.
^{c}Isolated yields.
^{d}Measured by chiral HPLC.
^{e}In the presence of 5 mol % Pd(OAc)₂.

TABLE 2
[Co(P6)]-Catalyzed Enantioselective Aziridination of Different Alkenes with
Trichloroethoxysulfonyl Azide (TcesN₃).^{a}

| | | | temp | yield | ee | |
|---|---|---|---|---|---|---|
| entry | olefin | aziridine | (° C.) | (%)^{b} | (%)^{c} | (α)^{d} |
| 1 | 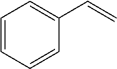 | 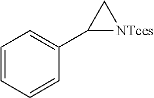 | 0 | 91 | 94 | (−) |
| 2 | 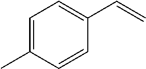 | 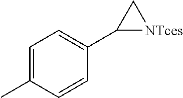 | 0 | 89 | 90 | (−) |
| 3^{f} | 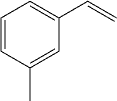 | 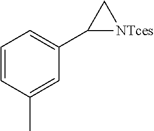 | RT | 85 | 82 | (−) |
| 4^{f} | 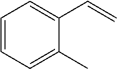 | 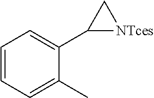 | RT | 86 | 84 | (−) |
| 5 | 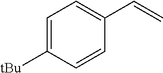 | 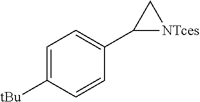 | 0 | 89 | 85 | (−) |
| 6 | 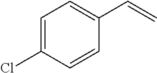 | 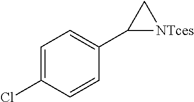 | 0 | 93 | 91 | (R)^{e} |
| 7 | 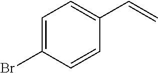 | 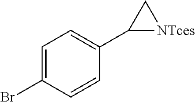 | 0 | 92 | 91 | (−) |
| 8 | 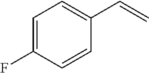 | 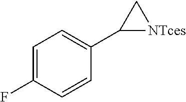 | 0 | 90 | 90 | (−) |
| 9 | 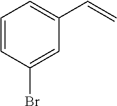 | 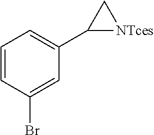 | 0 | 91 | 88 | (−) |
| 10 | 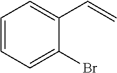 | 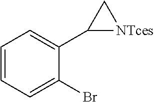 | 0 | 92 | 99 | (−) |
| 11 | 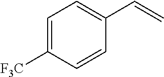 | 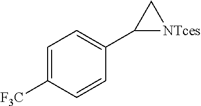 | 0 | 88 | 81 | (−) |
| 12 | 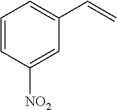 | 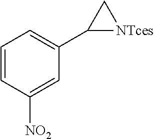 | 0 | 82 | 80 | (−) |
| 13 | 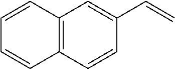 | 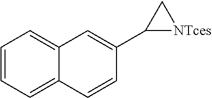 | 0 | 85 | 90 | (−) |
| 14^{f} | 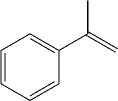 | 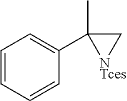 | RT | 48 | 80 | (−) |
| 15^{f} | 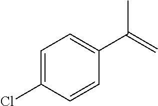 | 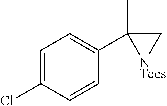 | RT | 43 | 80 | (−) |
| 16^{f} | 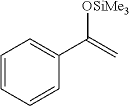 | 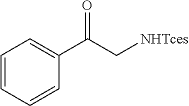 | 40 | 87 | — | — |
| 17^{g} | 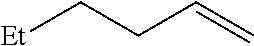 | 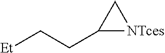 | 40 | 42 | 91 | (+) |
| 18^{g} | 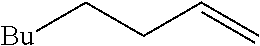 | 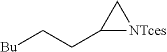 | 40 | 30 | 90 | (+) |
| 19^{f} | 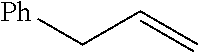 | 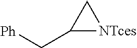 | 0 | 26 | 94 | (+) |
| 20^{h} | 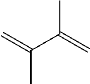 | 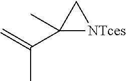 | RT | 53 | 87 | (−) |
| 21^{f} | 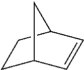 | 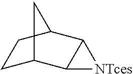 | 40 | 85 | — | — |
| 22^{f} | 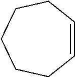 | 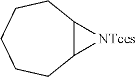 | 40 | >50ⁱ | — | — |

^{a}Performed in C₆H₅Cl using 5 mol % [Co(P6)] for 48 h under N₂ with 4 Å MS in the presence of 5 mol % Pd(OAc)₂: alkene:TcesN₃ = 5:1; [alkene] = 0.25M.
^{b}Isolated yields.
^{c}Measured by chiral HPLC.
^{d}Sign of optical rotation.
^{e}Determined by X-ray crystal structural analysis.
^{f}24 h without Pd(OAc)₂.
^{g}Using [Co(P5)]in CH₂Cl₂ for 48 h.
^{h}In CH₃CO₂C₂H₅.
ⁱPartial decomposition on silica gel.

TABLE S2
[Co(P6)]-Catalyzed Enantioselective Aziridination of Different Alkenes with
Trichloroethoxysulfonyl Azide (TcesN₃).^{a}

| | | | temp | yield | ee | |
|---|---|---|---|---|---|---|
| entry | olefin | aziridine | (° C.) | (%)^{b} | (%)^{c} | (α)^{d} |
| 1 2 3^{f} 4^{f,g} 5f | 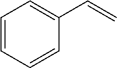 | 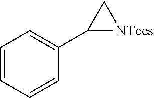 | RT 0 0 0 −10 | 90 69 91 60 59 | 88 94 94 94 96 | (−) |
| 6 7 8^{f} | 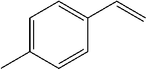 | 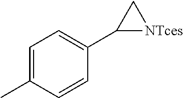 | RT 0 0 | 84 72 89 | 79 77 90 | (−) |
| 9 | 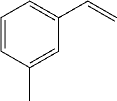 | 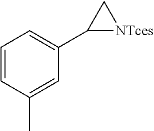 | RT | 85 | 82 | (−) |
| 10 | 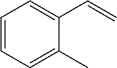 | 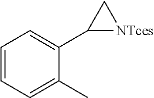 | RT | 86 | 84 | (−) |
| 11 12 13^{f} | 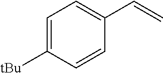 | 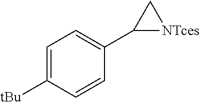 | RT 0 0 | 88 65 89 | 82 85 85 | (−) |
| 14 15 16^{f} | 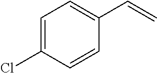 | 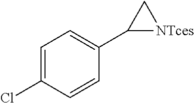 | RT 0 0 | 90 71 93 | 84 91 91 | (−) (R)^{e} |
| 17 18 19^{f} | 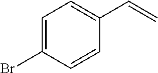 | 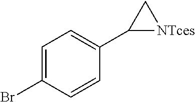 | RT 0 0 | 90 69 92 | 83 91 91 | (−) |
| 20 21 22 | 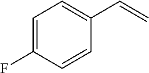 | 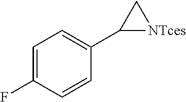 | RT 0 0 | 92 72 90 | 85 90 90 | (−) |
| 23 24 25^{f} | 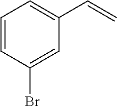 | 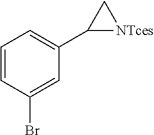 | RT 0 0 | 94 66 91 | 80 88 88 | (−) |
| 26 27 28^{f} | 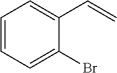 | 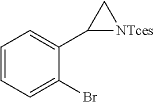 | RT 0 0 | 95 68 92 | 96 99 99 | (−) |
| 29 30 31^{f} | 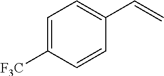 | 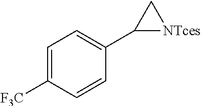 | RT 0 0 | 82 65 88 | 71 81 81 | (−) |
| 32 33 34^{f} | 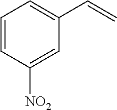 | 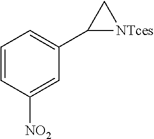 | RT 0 0 | 85 62 82 | 56 80 80 | (−) |
| 35 36 37^{f} | 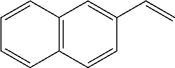 | 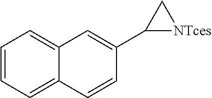 | RT 0 0 | 90 70 85 | 70 90 90 | (−) |
| 38 | 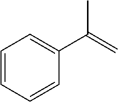 | 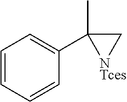 | RT | 48 | 80 | (−) |
| 39 | 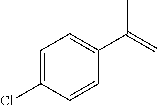 | 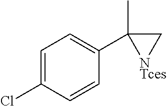 | RT | 43 | 80 | (−) |
| 40 | 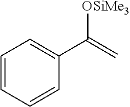 | 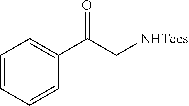 | 40 | 87 | — | — |
| 41^{h} 42^{f,h} | 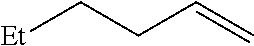 | 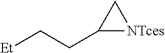 | 40 40 | 26 42 | 90 91 | (+) |
| 43^{f,h} | 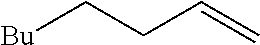 | 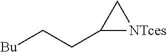 | 40 | 30 | 90 | (+) |
| 44 | 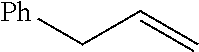 | 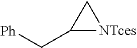 | 0 | 26 | 94 | (+) |
| 45ⁱ | 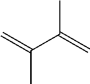 | 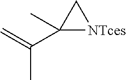 | RT | 53 | 87 | (−) |
| 46 | 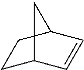 | 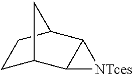 | 40 | 85 | — | — |
| 47 | 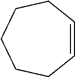 | 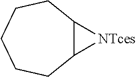 | 40 | >50^{j} | — | — |

^{a}Performed in C₆H₅Cl using 5 mol % [Co(P6)] for 24 h under N₂ with 4 Å MS: alkene:TcesN₃ = 5.0:1.0; [alkene] = 0.25M.
^{b}Isolated yields.
^{c}By chiral HPLC.
^{d}Sign of optical rotation.
^{e}Determined by X-ray crystal structural analysis.
^{f}Added 5 mol % Pd(OAc)₂ for 48 h.
^{g}Styrene:TcesN₃ = 1.0:1.1.
^{h}Using [Co(P5)]as catalyst in CH₂Cl₂ for 48 h.
ⁱIn CH₃CO₂C₂H₅.
^{j}Partial decomposition on silica gel.

TABLE 3
Reusability of [Co(P6)] for Asymmetric Aziridination.^{a}

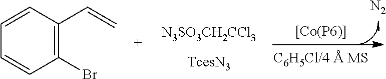

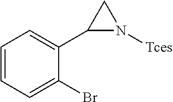

| | | temp | yield | ee |
|---|---|---|---|---|
| entry | cycle | (° C.) | (%)^{b} | (%)^{c} |
| 1 | [Co(P1)] | RT | 95 | 96 |
| 2 | [Co(P1)] | RT | 89 | 94 |
| 3 | [Co(P1)] | RT | 81 | 94 |

^{a}Performed in C₆H₅Cl using 5 mol % [Co(P6)] for 48 h under N₂ with 4 Å MS in the presence of 5 mol % Pd(OAc)₂: alkene:TcesN₃ = 5:1; [alkene] = 0.25M.
^{b}Isolated yields.
^{c}Measured by chiral HPLC.

### Supporting Examples and Details

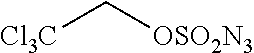

In summary, we have developed a highly asymmetric aziridination system based on [Co(P6)] and TcesN₃. This represents the first highly effective and enantioselective catalytic system for asymmetric aziridination of a broad range of simple olefins, without the need of additional functionalities in the substrates for secondary binding interactions. In addition to the common attributes associated with the use of azides as the nitrene sources, a simple protocol has been established to allow the recycling/reusing of the catalyst, further enhancing its potential for practical synthetic applications. This Co-catalyzed asymmetric aziridination with TcesN₃ complements the previous Cu/ArI=NTs-based systems, which are best suitable to internal olefins having additional functionalities.

Trichloroethoxysulfonyl Azide: ¹H NMR (400 MHz, CDCl₃): δ 4.74 (s, 2H). ¹³C NMR (100 MHz, CDCl₃): δ 92.3, 80.5. IR (neat, cm⁻¹): 2964, 2146, 1415, 1192, 1087, 995, 866, 784, 724, 622.

General Procedure for the Aziridination of Alkenes: An oven dried Schlenk tube, that was previously evacuated and backfilled with nitrogen gas, was charged with catalyst (0.05 mmol), Pd(OAc)₂ (0.05 mmol), and 4 Å MS (100 mg). The Schlenk tube was then evacuated and backfilled with nitrogen. The Teflon screw cap was replaced with a rubber septum and 0.5 ml of solvent was added followed by styrene (0.5 mmol) at room temperature, another portion of solvent at 0° C., then azide (0.1 mmol), and the remaining solvent (total 1 mL). The Schlenk tube was then purged with nitrogen for 1 minute and the rubber septum was replaced with the Teflon screw cap. The Schlenk tube was then placed at room temperature or 0° C. for 24-48 h. Following the completion of the reaction, the reaction mixture was purified by flash chromatography. The fractions containing product were collected and concentrated by rotary evaporation to afford the compound.

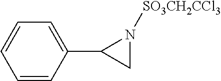

2-Phenyl-aziridine-1-sulfonic acid 2,2,2-trichloro-ethyl ester:² [α]²⁰D=−52.38 (c=0.31, CHCl₃, ee=94%). ¹H NMR (400 MHz, CDCl₃): δ 7.39-7.32 (m, 5H), 4.88 (d, 1H, J=10.9 Hz), 4.81 (d, 1H, J=10.9 Hz), 3.88 (dd, 1H, J=7.2, 4.6 Hz), 3.09 (d, 1H, J=7.2 Hz), 2.63 (d, 1H, J=4.7 Hz). ¹³C NMR (100 MHz, CDCl₃): δ 133.8, 128.9, 128.8, 126.5, 92.8, 79.6, 42.7, 37.5. IR (neat, cm⁻¹): 2925, 1365, 1182, 1094, 1008, 908, 880, 785, 716, 695, 622. HRMS (ESI) Calcd. for C₁₀H₁₀Cl₃NO₃S: 328.9447. Found 182.0280 (M⁺−OCH₂CCl₃). HPLC analysis: ee=96%. Welko-O 1 (99.5% hexanes: 0.5%-isopropanol, 1.0 mL/min): tₘᵢₙₒᵣ=19.0 min, tₘₐⱼₒᵣ=23.4 min.

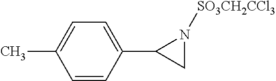

2-p-Tolyl-2-aziridine-1-sulfonic acid 2,2,2-trichloro-ethyl ester:² [α]²⁰D=−22.10 (c=0.75, CHCl₃, ee=90%). ¹H NMR (400 MHz, CDCl₃): δ 7.16 (s, 4H), 4.86 (d, 1H, J=10.8 Hz), 4.79 (d, 1H, J=10.8 Hz), 3.84 (dd, 1H, J=7.2, 4.8 Hz), 3.06 (d, 1H, J=6.8 Hz), 2.60 (d, 1H, J=4.8 Hz), 2.34 (s, 3H). ¹³C NMR (100 MHz, CDCl₃): δ 139.1, 130.9, 129.7, 126.7, 93.9, 79.9, 43.0, 37.6, 21.4. IR (neat, cm⁻¹): 2924, 2852, 1366, 1182, 1086, 1007, 917, 870, 788, 718. HRMS (ESI) ([M+H]⁺) Calcd. for C₁₁H₁₃Cl₃NO₃S: 343.9682. Found 343.9690. HPLC analysis: ee=90%. Welko-O 1 (99.5% hexanes: 0.5%-isopropanol, 1.0 mL/min): tₘᵢₙₒᵣ=19.1 min, tₘₐⱼₒᵣ=23.8 min.

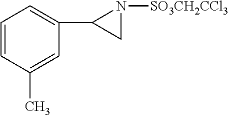

2-m-Tolyl-2-aziridine-1-sulfonic acid 2,2,2-trichloro-ethyl ester: [α]²⁰D=−49.99 (c=0.85, CHCl₃, ee=82%). ¹H NMR (400 MHz, CDCl₃): δ 7.26 (s, 4H), 4.81 (d, 1H, J=10.8 Hz), 3.83 (dd, 1H, J=7.2, 4.8 Hz), 3.06 (d, 1H, J=7.2 Hz), 2.60 (d, 1H, J=4.4 Hz), 2.34 (s, 3H). ¹³C NMR (100 MHz, CDCl₃): δ 138.8, 133.9, 129.9, 128.9, 127.4, 123.9, 93.1, 79.9, 43.0, 37.6, 21.6. IR (neat, cm⁻¹): 2921, 2851, 1463, 1378, 1186, 1090, 888, 721, 618. HRMS (ESI) ([M+H]⁺) Calcd. for C₁₁H₁₃Cl₃NO₃S: 343.9682. Found 343.9694. HPLC analysis: ee=82%. Welko-O 1 (99.5% hexanes: 0.5%-isopropanol, 1.0 mL/min): tₘᵢₙₒᵣ=18.7 min, tₘₐⱼₒᵣ=24.5 min.

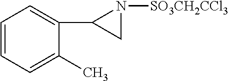

2-o-Tolyl-2-aziridine-1-sulfonic acid 2,2,2-trichloro-ethyl ester: [α]²⁰D=−24.81 (c=0.66, CHCl₃, ee=84%). ¹H NMR (400 MHz, CDCl₃): δ 7.24-7.27 (m, 4H), 4.90 (d, 1H, J=10.8 Hz), 4.84 (d, 1H, J=10.8 Hz), 3.99 (dd, 1H, J=7.2, 4.8 Hz), 3.08 (d, 1H, J=6.8 Hz), 2.57 (d, 1H, J=4.8 Hz), 2.44 (s, 3H). ¹³C NMR (100 MHz, CDCl₃): δ 137.3, 132.3, 130.6, 128.9, 126.5, 125.9, 93.1, 79.9, 41.5, 36.8, 19.3. IR (neat, cm⁻¹): 2926, 2854, 1364, 1180, 1095, 999, 921, 884, 846, 788, 723, 624. HRMS (ESI) ([M+H]⁺) Calcd. for C₁₁H₁₃Cl₃NO₃S: 343.9682. Found 343.9689. HPLC analysis: ee=84%. Chiralcel OD-H (98% hexanes: 2%-isopropanol, 1.0 mL/min): tₘᵢₙₒᵣ=14.0 min, tₘₐⱼₒᵣ=15.3 min.

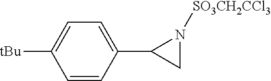

2-(4-tert-Butyl-phenyl)-aziridine-1-sulfonic acid 2,2,2-trichloro-ethyl ester: [α]²⁰D=−17.08 (c=1.51, CHCl₃, ee=85%). ¹H NMR (400 MHz, CDCl₃): δ 7.40 (d, 2H, J=8.4 Hz), 7.25 (d, 2H, J=8.4 Hz), 4.89 (d, 1H, J=10.8 Hz), 4.82 (d, 1H, J=10.8 Hz), 3.87 (dd, 1H, J=7.2, 4.8 Hz), 3.08 (d, 1H, J=7.2 Hz), 2.63 (d, 1H, J=4.4 Hz), 1.34 (s, 9H). ¹³C NMR (100 MHz, CDCl₃): δ 152.4, 130.9, 126.6, 125.9, 93.1, 79.9, 42.9, 37.6, 34.9, 32.2, 31.5, 30.8. IR (neat, cm⁻¹): 2922, 2852, 1493, 1377, 1260, 1183, 1389, 1385, 1182, 1089, 1018, 801, 725. HRMS (ESI) ([M+H]⁺) Calcd. for C₁₄H₁₉Cl₃NO₃S: 386.0152. Found 386.0159. HPLC analysis: ee=85%. Welko-O 1 (99.5% hexanes: 0.5%-isopropanol, 1.0 mL/min): tₘᵢₙₒᵣ=15.8 min, tₘₐⱼₒᵣ=18.5 min.

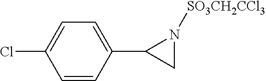

2-(4-Chloro-phenyl)-aziridine-1-sulfonic acid 2,2,2-trichloro-ethyl ester:² [α]²⁰D=−56.13 (c=0.46, CHCl₃, ee=91%). ¹H NMR (400 MHz, CDCl₃): δ 7.34 (d, 2H, J=8.4 Hz), 7.23 (d, 2H, J=8.4 Hz), 4.86 (d, 1H, J=10.8 Hz), 4.80 (d, 1H, J=10.8 Hz), 3.83 (dd, 1H, J=7.2, 4.8 Hz), 3.08 (d, 1H, J=7.2 Hz), 2.57 (d, 1H, J=4.8 Hz). ¹³C NMR (100 MHz, CDCl₃): δ 135.1, 132.6, 129.3, 128.1, 92.9, 79.9, 42.3, 37.8. IR (neat, cm⁻¹): 2922, 2851, 1366, 1182, 1085, 1007, 915, 868, 792, 716. HRMS (ESI) ([M]⁺) Calcd. for C₁₀H₉Cl₄NO₃S: 362.9057. Found 362.9077. HPLC analysis: ee=91%. Welko-O 1 (99.5% hexanes: 0.5%-isopropanol, 1.0 mL/min): tₘᵢₙₒᵣ=20.4 min, tₘₐⱼₒᵣ=25.8 min.

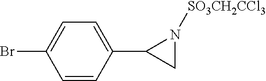

2-(4-Bromo-phenyl)-aziridine-1-sulfonic acid 2,2,2-trichloro-ethyl ester: [α]²⁰D=−51.64 (c=0.57, CHCl₃, ee=91%). ¹H NMR (400 MHz, CDCl₃): δ 7.49 (d, 2H, J=8.4 Hz), 7.17 (d, 2H, J=8.4 Hz), 4.86 (d, 1H, J=11.2 Hz), 4.80 (d, 1H, J=11.2 Hz), 3.82 (dd, 1H, J=7.2, 4.8 Hz), 3.08 (d, 1H, J=7.2 Hz), 2.57 (d, 1H, J=4.4 Hz). ¹³C NMR (100 MHz, CDCl₃): δ 133.2, 132.2, 128.4, 123.2, 92.9, 79.9, 42.3, 37.7. IR (neat, cm⁻¹): 2926, 2853, 1365, 1179, 1090, 1008, 925, 889, 862, 789, 723. HRMS (ESI) ([M]⁺) Calcd. for C₁₀H₉BrCl₃NO₃S: 406.8552. Found 406.8580. HPLC analysis: ee=91%. Welko-O 1 (99.5% hexanes: 0.5%-isopropanol, 1.0 mL/min): tₘᵢₙₒᵣ=22.6 min, tₘₐⱼₒᵣ=28.7 min.

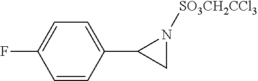

2-(4-Fluoro-phenyl)-aziridine-1-sulfonic acid 2,2,2-trichloro-ethyl ester: [α]²⁰D=−46.12 (c=0.66, CHCl₃, ee=90%). ¹H NMR (400 MHz, CDCl₃): δ 7.28 (d, 2H, J=8.8 Hz), 7.06 (d, 2H, J=8.4 Hz), 4.86 (d, 1H, J=10.8 Hz), 4.80 (d, 1H, J=10.8 Hz), 3.85 (dd, 1H, J=7.2, 4.4 Hz), 3.07 (d, 1H, J=7.2 Hz), 2.57 (d, 1H, J=4.8 Hz). ¹³C NMR (100 MHz, CDCl₃): CDCl₃): δ 164.5, 161.9, 129.8, 128.6, 128.5, 116.2, 115.9, 93.0, 79.9, 42.3, 37.7. IR (neat, cm⁻¹): 2925, 2854, 1516, 1367, 1182, 1086, 1008, 975, 919, 871, 837, 793, 717. HRMS (ESI) ([M]⁺) Calcd. for C₁₀H₉Cl₃FNO₃S: 346.9353. Found 346.9370. HPLC analysis: ee=90%. Welko-O 1 (99.5% hexanes: 0.5%-isopropanol, 1.0 mL/min): tₘᵢₙₒᵣ=17.4 min, tₘₐⱼₒᵣ=21.0 min.

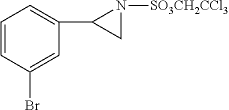

2-(3-Bromo-phenyl)-aziridine-1-sulfonic acid 2,2,2-trichloro-ethyl ester: [α]²⁰D=−54.87 (c=0.65, CHCl₃, ee=88%). ¹H NMR (400 MHz, CDCl₃): δ 7.43-7.37 (m, 2H), 7.19-7.17 (m, 2H), 4.82 (d, 1H, J=10.8 Hz), 4.66 (d, 1H, J=10.8 Hz), 3.77 (dd, 1H, J=7.2, 4.4 Hz), 3.02 (d, 1H, J=7.2 Hz), 2.52 (d, 1H, J=4.8 Hz). ¹³C NMR (100 MHz, CDCl₃): δ 136.4, 132.3, 130.6, 129.7, 125.6, 123.1, 92.9, 79.9, 41.9, 37.7. IR (neat, cm⁻¹): 2925, 2853, 1464, 1369, 1181, 1088, 1009, 925, 854, 782, 725, 684. HRMS (ESI) ([M]⁺) Calcd. for C₁₀H₉BrCl₃NO₃S: 407.8641. Found 407.8652. HPLC analysis: ee=88%. Welko-O 1 (99.5% hexanes: 0.5%-isopropanol, 1.0 mL/min): tₘᵢₙₒᵣ=20.2 min, tₘₐⱼₒᵣ=25.2 min.

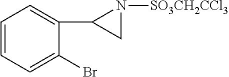

2-(2-Bromo-phenyl)-aziridine-1-sulfonic acid 2,2,2-trichloro-ethyl ester:² [α]²⁰D=−49.70 (c=0.56, CHCl₃, ee=99%). ¹H NMR (400 MHz, CDCl₃): δ 7.52 (d, 1H, J=8.0 Hz), 7.28-7.12 (m, 3H), 4.85 (d, 1H, J=10.8 Hz), 4.80 (d, 1H, J=10.8 Hz), 4.08 (dd, 1H, J=7.2, 4.8 Hz), 3.06 (d, 1H, J=7.2 Hz), 2.43 (d, 1H, J=4.4 Hz). ¹³C NMR (100 MHz, CDCl₃): δ 133.9, 132.9, 130.3, 127.9, 127.8, 123.6, 93.1, 79.9, 43.0, 37.6. IR (neat, cm⁻¹): 2923, 2854, 1464, 1372, 1260, 1183, 1096, 1002, 917, 788, 755, 724, 623. HRMS (ESI) ([M]⁺) Calcd. for C₁₀H₉BrCl₃NO₃S: 406.8552. Found 406.8535. HPLC analysis: ee=99%. Chiralcel OJ-H (95% hexanes: 5%-isopropanol, 1 mL/min): tₘₐⱼₒᵣ=13.3 min, tₘᵢₙₒᵣ=16.4 min.

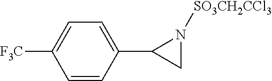

2-(4-Trifluoromethyl-phenyl)-aziridine-1-sulfonic acid 2,2,2-trichloro-ethyl ester: [α]²⁰D=−42.07 (c=0.80, CHCl₃, ee=81%). ¹H NMR (400 MHz, CDCl₃): δ 7.63 (d, 2H, J=8.0 Hz), 7.40 (d, 2H, J=8.0 Hz), 4.88 (d, 1H, J=10.8 Hz), 4.82 (d, 1H, J=10.8 Hz), 3.91 (dd, 1H, J=7.2, 4.4 Hz), 3.12 (d, 1H, J=7.2 Hz), 2.59 (d, 1H, J=4.4 Hz). ¹³C NMR (100 MHz, CDCl₃): δ 138.2, 131.5, 131.3, 127.2, 126.1, 126.0, 125.4, 92.9, 79.9, 42.0, 37.9. IR (neat, cm⁻¹): 2925, 2854, 1382, 1324, 1182, 1121, 1068, 1009, 920, 850, 783, 724. HRMS (ESI) ([M+H]⁺) Calcd. for C₁₁H₁₀Cl₃F₃NO₃S: 397.9395. Found 397.9390. HPLC analysis: ee=81%. Welko-O 1 (99.5% hexanes: 0.5%-isopropanol, 1.0 mL/min): tₘᵢₙₒᵣ=16.4 min, tₘₐⱼₒᵣ=19.5 min.

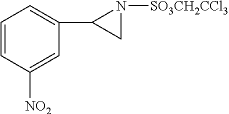

2-(3-Nitro-phenyl)-aziridine-1-sulfonic acid 2,2,2-trichloro-ethyl ester:² [α]²⁰D=−101.66 (c=0.32, CHCl₃, ee=80%). ¹H NMR (400 MHz, CDCl₃): δ 8.22 (d, 1H, J=8.0 Hz), 8.15 (s, 1H), 7.66 (d, 1H, J=8.0 Hz), 7.58 (t, 1H, J=8.0 Hz), 4.90 (d, 1H, J=10.8 Hz), 4.84 (d, 1H, J=10.8 Hz), 3.95 (dd, 1H, J=7.2, 4.4 Hz), 3.15 (d, 1H, J=7.2 Hz), 2.63 (d, 1H, J=4.8 Hz). ¹³C NMR (100 MHz, CDCl₃): δ 148.8, 136.5, 122.9, 130.2, 124.1, 121.7, 92.9, 80.0, 41.6, 37.9. IR (neat, cm⁻¹): 2922, 2853, 1534, 1368, 1345, 1182, 1010, 939, 874, 787, 720, 648. calcd for C₁₀H₉Cl₃N₂O₅S 373.9298. Found 227.0130 (M⁺−OCH₂CCl₃). HPLC analysis: ee=80%. Welko-O 1 (98% hexanes: 2%-isopropanol, 1.0 mL/min): tₘᵢₙₒᵣ=29.8 min, tₘₐⱼₒᵣ=32.2 min.

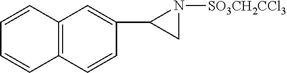

2-Napthalene-2yl-aziridine-1-sulfonic acid 2,2,2-trichloro-ethyl ester: [α]²⁰D=−86.69 (c=0.27, CHCl₃, ee=90%). ¹H NMR (400 MHz, CDCl₃): δ 7.85-7.81 (m, 4H), 7.52-7.50 (m, 2H), 7.33-7.31 (m, 1H), 4.90 (d, 1H, J=10.8 Hz), 4.83 (d, 1H, J=10.8 Hz), 4.04 (dd, 1H, J=7.2, 4.4 Hz), 3.16 (d, 1H, J=7.2 Hz), 2.72 (d, 1H, J=4.8 Hz). ¹³C NMR (100 MHz, CDCl₃): δ 133.6, 122.2, 131.4, 129.0, 128.1, 128.0, 126.9, 126.9, 127.8, 123.5, 93.1, 79.9, 43.3, 37.7. IR (neat, cm⁻¹): 2922, 2852, 1364, 1260, 1180, 1090, 1003, 861, 788, 721, 643. HRMS (ESI) ([M+H]⁺) Calcd. for C₁₄H₁₃Cl₃NO₃S: 379.9682. Found 379.9694. HPLC analysis: ee=90%. Welko-O 1 (98% hexanes: 2%-isopropanol, 1.0 mL/min): tₘᵢₙₒᵣ=19.6 min, tₘₐⱼₒᵣ=28.1 min.

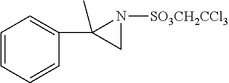

2-Phenyl-1-Methyl-aziridine-1-sulfonic acid 2,2,2-trichloro-ethyl ester: [α]²⁰D=−1.68 (c=0.26, CHCl₃, ee=80%). ¹H NMR (400 MHz, CDCl₃): δ 7.38-7.19 (m, 5H), 4.81 (s, 2H), 3.00 (s, 1H), 2.77 (s, 1H), 1.95 (s, 3H). IR (neat, cm⁻¹): 2922, 1365, 1182, 1094, 1008, 908, 880, 785, 716, 695, 622. HRMS (ESI) ([M+H]⁺) Calcd. for C₁₁H₁₃Cl₃NO₃S: 343.9682. Found 343.9630. HPLC analysis: ee=80%. Chiralcel OD-H (98.5% hexanes: 1.5%-isopropanol, 0.8 mL/min): tₘᵢₙₒᵣ=16.2 min, tₘₐⱼₒᵣ=17.3 min.

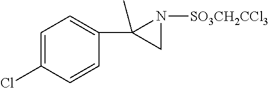

4-Chloro-2-Phenyl-1-Methyl-aziridine-1-sulfonic acid 2,2,2-trichloro-ethyl ester: [α]²⁰D=−1.92 (c=0.16, CHCl₃, ee=80%). ¹H NMR (400 MHz, CDCl₃): δ 7.29 (s, 4H), 4.77 (s, 2H), 2.97 (s, 1H), 2.71 (s, 1H), 1.89 (s, 3H). ¹³C NMR (100 MHz, CDCl₃): δ 138.22, 134.54, 129.94, 129.11, 128.13, 93.26, 79.71, 50.74, 43.42, 20.82. IR (neat, cm⁻¹): 2922, 1365, 1182, 1094, 1008, 908, 880, 785, 716, 695, 622. HRMS (ESI) ([M+H]⁺) Calcd. for C₁₁H₁₂Cl₄NO₃S: 377.9293. Found 377.9278. HPLC analysis: ee=80%. Chiralcel OD-H (98.5% hexanes: 1.5%-isopropanol, 0.8 mL/min): tₘᵢₙₒᵣ=15.4 min, tₘₐⱼₒᵣ=19.6 min.

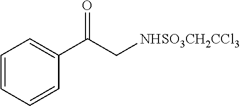

¹H NMR (400 MHz, CDCl₃): δ 7.94 (d, 2H, J=7.28 Hz), 7.66 (t, 1H, J=7.2 Hz), 7.51 (t, 2H, J=7.6 Hz), 5.94 (bs, 1H), 4.70 (d, 1H, J=4.0 Hz), 4.65 (s, 2H), 3.16 (d, 1H, J=7.2 Hz), 2.72 (d, 1H, J=4.8 Hz). ¹³C NMR (100 MHz, CDCl₃): δ 192.27, 135.05, 133.66, 129.39, 128.24, 93.49, 78.61, 49.47. IR (neat, cm⁻¹): 3280, 2958, 2922, 2852, 1686, 1448, 1409, 1371, 1313, 1234, 1178, 1079, 1014, 837, 747, 717, 620. HRMS (ESI) ([M+H]⁺) Calcd. for C₁₀H₁₁Cl₃NO₄S: 345.9679. Found 345.9694.

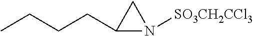

2-Butyl-aziridine-1-sulfonic acid 2,2,2-trichiro-ethyl ester³: [α]²⁰D=+ 35.09 (c=0.12, CHCl₃, ee=91%). ¹H NMR (400 MHz, CDCl₃): δ 4.90 (d, 1H, J=10.8 Hz), 4.83 (d, 1H, J=10.8 Hz), 2.87 (m, 1H), 2.72 (d, 1H, J=7.2 Hz, 2.25 (d, 1H, J=4.8 Hz), 1.62-1.33 (m, 6H), 0.92 (t, 3H). ¹³C NMR (100 MHz, CDCl₃): δ 93.0, 79.6, 42.6, 35.7, 31.0, 28.8, 22.4, 14.2. IR (neat, cm⁻¹): 2958, 2852, 1364, 1260, 1180, 1090, 1013, 861, 788, 721, 643. HRMS (ESI) ([M]⁺) Calcd. for C₄H₁₄Cl₃NO₃S: 308.9760. Found 308.9786. HPLC analysis: ee=91%. Welko-O 1 (99% hexanes: 1%-isopropanol, 1.0 mL/min): tₘₐⱼₒᵣ=8.2 min, tₘᵢₙₒᵣ=9.6 min.

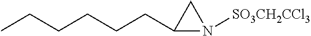

2-Hexyl-aziridine-1-sulfonic acid 2,2,2-trichiro-ethyl ester³:[α]²⁰D=+ 45.09 (c=0.10, CHCl₃, ee=90%). ¹H NMR (400 MHz, CDCl₃): δ 4.91 (d, 1H, J=10.8 Hz), 4.84 (d, 1H, J=10.8 Hz), 2.89 (m, 1H), 2.74 (d, 1H, J=7.2 Hz, 2.26 (d, 1H, J=4.8 Hz), 1.65-1.31 (m, 10H), 0.91 (t, 3H). ¹³C NMR (100 MHz, CDCl₃): δ 93.0, 79.6, 42.6, 35.7, 31.0, 28.8, 22.4, 14.2. IR (neat, cm⁻¹): 2958, 2852, 1364, 1260, 1180, 1090, 1013, 861, 788, 721, 643. HRMS (ESI) ([M+H]⁺) Calcd. for C₁₀H₁₉Cl₃NO₃S: 338.0152. Found 338.0140. HPLC analysis: ee=91%. Welko-O 1 (99% hexanes: 1%-isopropanol, 1.0 mL/min): tₘₐⱼₒᵣ=8.2 min, tₘᵢₙₒᵣ=9.6 min.

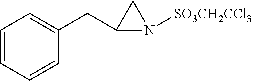

[α]²⁰D=+ 56.21 (c=0.16, CHCl₃, ee=94%). ¹H NMR (400 MHz, CDCl₃): δ 7.56-7.22 (m, 5H), 4.67 (d, 1H, J=10.8 Hz), 4.54 (d, 1H, J=10.8 Hz), 3.15-3.09 (m, 1H), 2.96-2.84 (m, 1H), 2.77 (d, 1H, J=7.2 Hz), 2.33 (d, 1H, J=4.8 Hz). ¹³C NMR (100 MHz, CDCl₃): δ 136.1, 129.06, 129.01, 127.48, 92.6, 79.62, 42.55, 37.38, 35.23. IR (neat, cm⁻¹): 2958, 2852, 1364, 1260, 1180, 1090, 1013, 861, 788, 721, 643. HRMS (ESI) ([M+H]⁺) Calcd. for C₁₁H₁₃Cl₃NO₃S: 342.9603. Found 342.9615. HPLC analysis: ee=94%. Chiralcel OJ-H (95% hexanes: 5%-isopropanol, 0.8 mL/min): tₘᵢₙₒᵣ=34.6 min, tₘᵢₙₒᵣ=42.6 min.

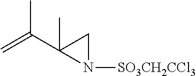

[α]²⁰D=−6.03 (c=0.16, CHCl₃, ee=87%). ¹H NMR (400 MHz, CDCl₃): 5.04 (s, 1H), 4.98 (s, 1H), 4.78 (s, 2H), 2.79 (s, 1H), 2.63 (s, 1H), 1.80 (s, 3H), 1.73 (s, 3H). ¹³C NMR (100 MHz, CDCl₃): δ 143.3, 114.5, 93.1, 79.6, 52.8, 42.8, 19.1, 18.1. IR (neat, cm⁻¹): 2940, 2852, 1364, 1260, 1180, 1090, 1013, 861, 788, 721, 643. HRMS (ESI) ([M+H]⁺) Calcd. for C₈H₁₃Cl₃NO₃S: 307.9682. Found 307.9652. HPLC analysis: ee=87%. Welko-O 1 (99.5% hexanes: 0.5%-isopropanol, 1.0 mL/min): tₘᵢₙₒᵣ=9.5 min, tₘₐⱼₒᵣ=10.4 min.

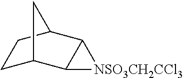

¹H NMR (400 MHz, CDCl₃): δ 4.75 (s, 2H), 3.03 (s, 2H), 2.54 (s, 2H), 1.53 (d, 2H, J=7.2 Hz), 1.46 (d, 1H, J=10.0 Hz), 1.28 (m, 2H), 0.84 (d, 2H, J=10.0 Hz). ¹³C NMR (100 MHz, CDCl₃): δ 93.40, 79.60, 43.87, 36.04, 28.28, 28.24, 25.52. IR (neat, cm⁻¹): 2985, 2880, 1363, 1288, 1175, 1092, 1012, 979, 917, 870, 789, 723, 621. HRMS (ESI) ([M+H]⁺) Calcd. for C₉H₁₃Cl₃NO₃S: 319.9682. Found 319.9689.

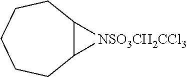

N-(2, 2, 2-Trichloroethoxylsulfonyl)-8-aza-bicylo[5,1,0]octane:^{3 1}H NMR (400 MHz, CDCl₃): δ 4.80 (s, 2H), 3.10 (m, 2H), 2.06-1.88 (m, 4H), 1.65-1.47 (m, 5H), 1.23 (m, 1H). ¹³C NMR (100 MHz, CDCl₃): δ 93.31, 79.56, 46.64, 31.06, 28.14, 25.40. IR (neat, cm⁻¹): 2960, 2879, 1365, 1270, 1180, 1081, 1030, 869, 779, 709, 627. HRMS (ESI) ([M+H]⁺) Calcd. for C₉H₁₅Cl₃NO₃S: 321.9839. Found 321.9850.

What is claimed is:

1. A process for the asymmetric aziridination of an alkene, the process comprising treating the alkene with a sulfonyl azide in the presence of a cobalt(II) porphyrin complex, the sulfonyl azide having the formula 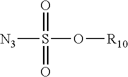
wherein R₁₀ is hydrocarbyl, substituted hydrocarbyl, or heterocyclo.

2. The process of claim 1 wherein R₁₀ is trihaloakyl.

3. The process of claim 1 wherein R₁₀ is trichloroethyl.

4. The process of claim 3 wherein the cobalt(II) complex is a D₂-symmetric chiral porphyrin.

5. The process of 1 wherein the cobalt(II) complex is a D₂-symmetric chiral porphyrin selected from the group consisting of

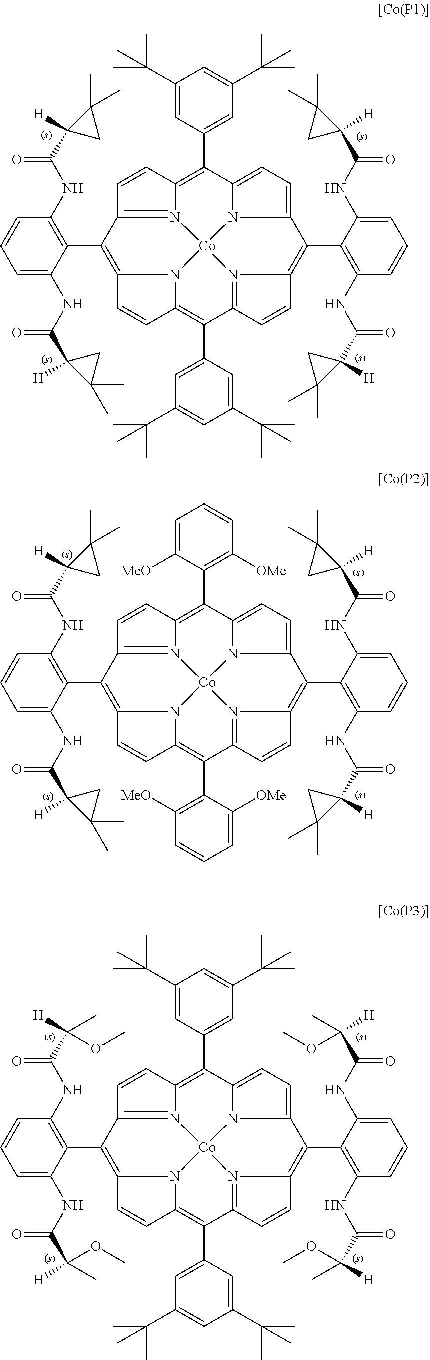

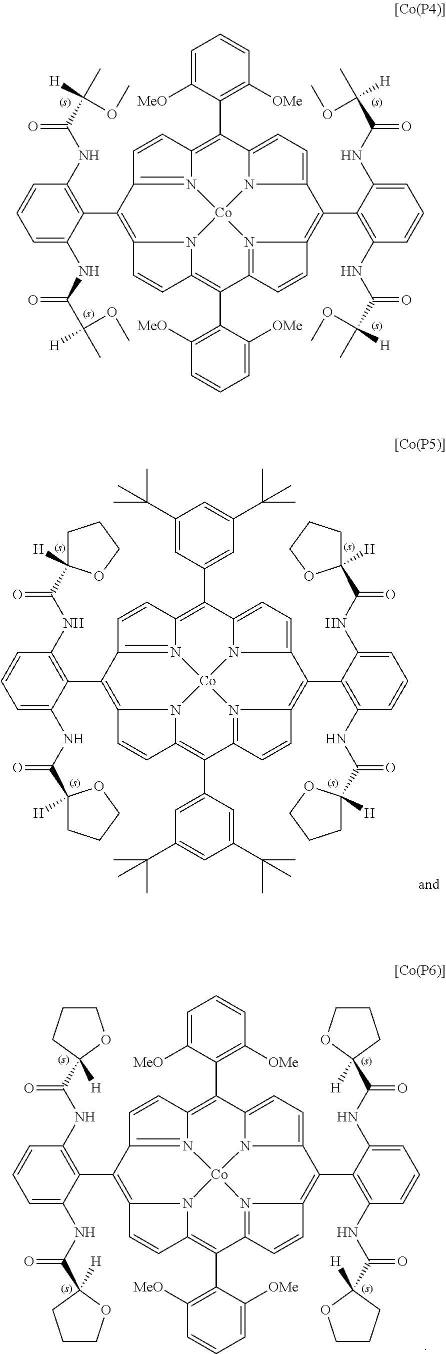

6. The process of 3 wherein the cobalt(II) complex is a D₂-symmetric chiral porphyrin selected from the group consisting of

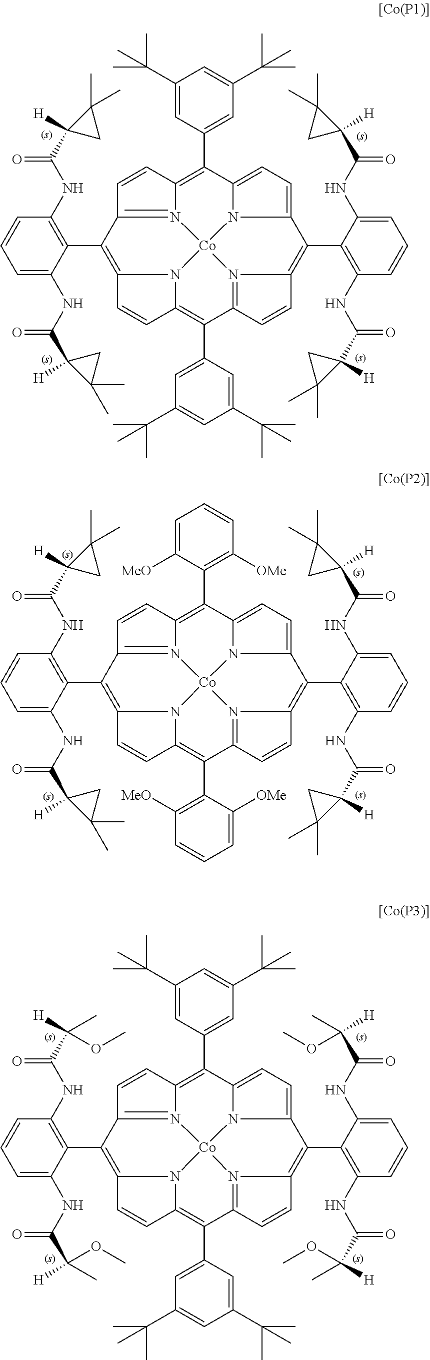

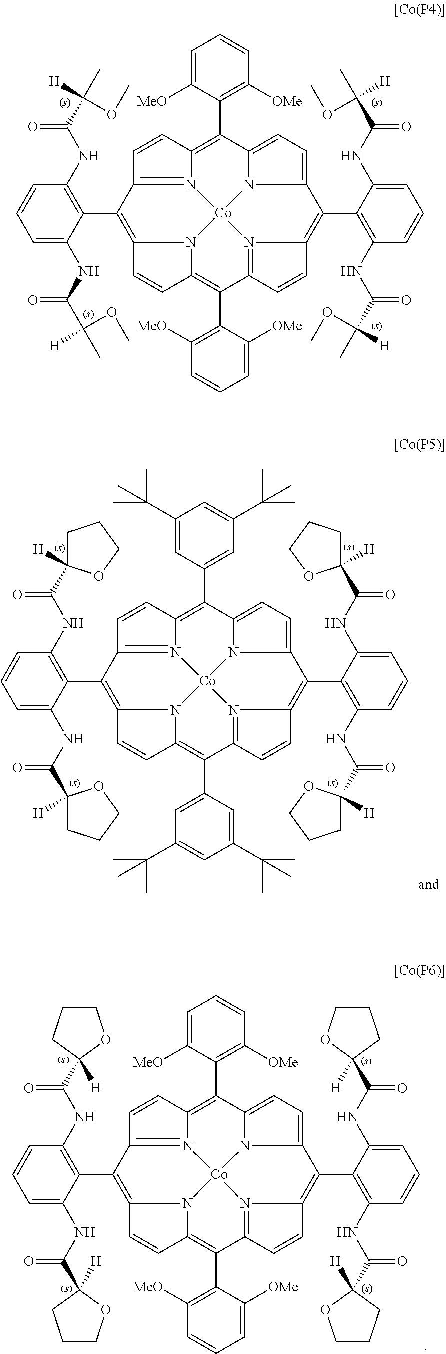

7. The process of claim 1 wherein the alkene corresponds to Formula 1 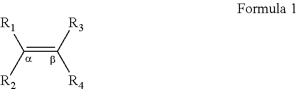
wherein R₁, R₂, R₃, and R₄ are independently hydrogen, hydrocarbyl, substituted hydrocarbyl, heterocyclo or an electron withdrawing group.

8. The process of claim 3 wherein the alkene corresponds to Formula 1 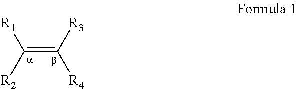
wherein R₁, R₂, R₃, and R₄ are independently hydrogen, hydrocarbyl, substituted hydrocarbyl, heterocyclo or an electron withdrawing group.

9. The process of claim 5 wherein the alkene corresponds to Formula 1 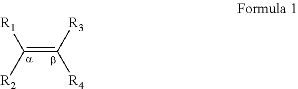
wherein R₁, R₂, R₃, and R₄ are independently hydrogen, hydrocarbyl, substituted hydrocarbyl, heterocyclo or an electron withdrawing group.

10. The process of claim 6 wherein the alkene corresponds to Formula 1 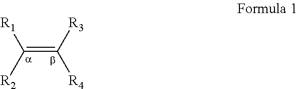
wherein R₁, R₂, R₃, and R₄ are independently hydrogen, hydrocarbyl, substituted hydrocarbyl, heterocyclo or an electron withdrawing group.

11. The process of claim 1 wherein the alkene corresponds to Formula 1 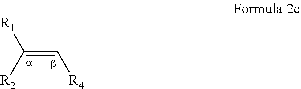
wherein R₁, R₂, and R₄ are independently hydrogen, hydrocarbyl, substituted hydrocarbyl, or heterocyclo.

12. The process of claim 3 wherein the alkene corresponds to Formula 1 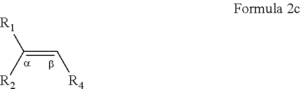
wherein R₁, R₂, and R₄ are independently hydrogen, hydrocarbyl, substituted hydrocarbyl, or heterocyclo.

13. The process of claim 5 wherein the alkene corresponds to Formula 1 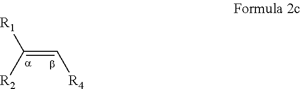
wherein R₁, R₂, and R₄ are independently hydrogen, hydrocarbyl, substituted hydrocarbyl, or heterocyclo.

14. The process of claim 6 wherein the alkene corresponds to Formula 1 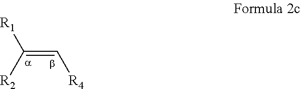
wherein R₁, R₂, and R₄ are independently hydrogen, hydrocarbyl, substituted hydrocarbyl, or heterocyclo.

15. The process of claim 1 wherein the alkene corresponds to Formula 3(a): 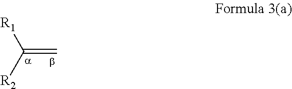
wherein R₁ and R₂ are independently hydrogen, hydrocarbyl, substituted hydrocarbyl, or heterocyclo.

16. The process of claim 3 wherein the alkene corresponds to Formula 3(a): 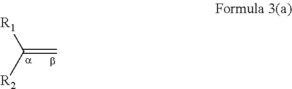
wherein R₁ and R₂ are independently hydrogen, hydrocarbyl, substituted hydrocarbyl, or heterocyclo.

17. The process of claim 5 wherein the alkene corresponds to Formula 3(a): 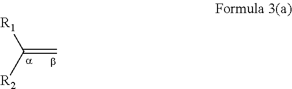
wherein R₁ and R₂ are independently hydrogen, hydrocarbyl, substituted hydrocarbyl, or heterocyclo.

18. The process of claim 6 wherein the alkene corresponds to Formula 3(a): 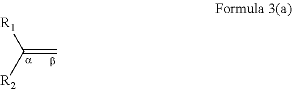
wherein R₁ and R₂ are independently hydrogen, hydrocarbyl, substituted hydrocarbyl, or heterocyclo.

19. The process of claim 1 wherein the alkene corresponds to Formula 3(a): 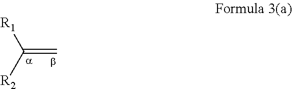
wherein R₁ and R₂ and the α-carbon to which they are bonded, in combination, comprise a carbocyclic or heterocyclic ring system.

20. Trichloroethoxysulfonyl azide.
